(19) United States
(12) Patent Application Publication
(10) Pub. No.: US 2024/0417778 A1
(43) Pub. Date: Dec. 19, 2024

(54) Methods for Nucleic Acid Cleavage

(71) Applicant: Cambridge Enterprise Limited, Cambridge, Cambridgeshire (GB)

(72) Inventors: Gonçalo Bernardes, Cambridge (GB); Sigitas Mikutis, Cambridge (GB); Anna Sib, Cambridge (GB)

(21) Appl. No.: 18/702,310

(22) PCT Filed: Oct. 28, 2022
(86) PCT No.: PCT/EP2022/080220

§ 371 Date: Apr. 17, 2024

(30) Foreign Application Priority Data
Oct. 28, 2021 (GB) 2115540.3

(51) Int. Cl. C12Q 1/6806

(52) U.S. Cl. CPC C12Q 1/6806

(57) ABSTRACT

The invention provides a method for cleaving a target nucleic acid molecule. The method comprises contacting the target nucleic acid molecule with a bifunctional molecule of formula (I), C-L-B, where —C is a cleavage group that is imidazole, optionally substituted with 1 to 3 C₁₋₆ alkyl groups, -L- is a linker and —B is a non-covalent binding group, such that the bifunctional molecule non-covalently binds to the target nucleic acid molecule, and allowing the bifunctional molecule to cleave the target nucleic acid molecule bound thereto. The invention also provides a method of identifying a secondary or tertiary structure within a target nucleic acid, as well as a bifunctional molecule and a bifunctional molecule for use in a method of treatment.

### RELATED APPLICATION

The present application is a U.S. national phase filing of PCT Application No. PCT/EP2022/080220, filed on Oct. 28, 2022, which claims the benefit of and priority to GB 2115540.3, filed on 28 Oct. 2021 (28/10/2021), the contents of which are hereby incorporated by reference in their entirety.

### INCORPORATION-BY-REFERENCE OF MATERIAL ELECTRONICALLY SUBMITTED

A Sequence Listing is provided herewith as a Sequence Listing XML, “MEWE-122_Sequence listing” created on Jan. 10, 2023, and having a size of 29,696 bytes. The contents of the Sequence Listing XML are incorporated herein by reference in their entirety.

### FIELD OF THE INVENTION

The present invention relates to methods for the non-enzymatic cleavage of target nucleic acids, for example for use in epigenomic and epitranscriptomic mapping, as well as in therapy, for example anti-microbial and anti-viral therapy.

### BACKGROUND

Advances in nucleic acid manipulation and editing technologies have revolutionized the way biological research is conducted. RNA interference and shRNA expression systems have proven invaluable for target validation as well as elucidation of the role particular genes play in molecular diseases (Zamore et al., 2000). More recently, CRISPR-based technologies have enhanced the ability to manipulate DNA (Gasiunas et al., 2012; Jinek et al., 2012) and RNA (Cox et al. 2017) even further, empowering simpler systems such as gene knock-out cells and enabling large and elaborate CRISPR-Cas9-based genetic screening approaches (Tzelepis et al., 2016). However, the most commonly used nucleic acid manipulation technologies are genetic and it is challenging if not outright impossible to apply them to more complex biological systems such as rare populations, animal models and whole tissues, and yet even harder to develop therapeutics based on these technologies (Bobbin et al, 2016). Therefore, there is a need to develop new small molecule-based technologies, which would enable manipulation of nucleic acids in yet unexplored contexts.

Mikutis et al., 2020 describes a small molecule “click degrader” that can be covalently attached to an RNA species through click-chemistry and can then cleave the attached RNA molecule. The authors describe a methylation CLICK degradation sequencing method (meCLICK-Seq) for identifying the presence of N⁶-methyladenosine (m⁶A) in an RNA sequence. The method hijacks an RNA methyltransferase to introduce an alkyne moiety, instead of a methyl group, on RNA. A subsequent copper(I)-catalyzed azide-alkyne cycloaddition reaction incorporates the click-degrader molecule, leading to RNA cleavage.

The method identifies methylated transcripts, determines RNA methylase specificity, and reliably maps modification sites in intronic and intergenic regions.

As the click degrader molecules are covalently incorporated into the target RNA, they can only be used to degrade RNA species which can be edited to contain a suitable click-reactive group (typically an alkyne). Moreover, the required editing of the RNA limits the application of the technology to therapeutics.

The present invention has been devised in light of the above considerations.

### SUMMARY OF THE INVENTION

The present invention relates to the finding that a bifunctional molecule, known herein as a degrader, can be used as a catalytic agent to non-covalently bind to and cleave a target nucleic acid molecule. The degraders disclosed herein bind to a target nucleic acid through non-covalent interactions. Surprisingly, the inventors have found that non-covalent binding is sufficient to enable the selective degradation of the target nucleic acid. Accordingly, the degraders do not require incorporation of a click-reactive group into the target nucleic acid.

The selective cleavage of target nucleic acid molecules using the degraders described herein may be useful in epigenetic and epitranscriptomic analysis, bifunctional mapping, as well as in therapy, for example anti-bacterial and anti-viral therapy, and additionally anti-cancer.

Accordingly, in a first aspect of the invention there is provided a method for cleaving a target nucleic acid molecule, the method comprising:
contacting the target nucleic acid molecule with a bifunctional molecule of formula (I):

C-L-B (I)
where —C is a cleavage group that is imidazole, optionally substituted with 1 to 3 C₁₋₆ alkyl groups, -L- is a linker and —B is a non-covalent binding group, such that the bifunctional molecule non-covalently binds to the target nucleic acid molecule, and;
allowing the bifunctional molecule to cleave the target nucleic acid molecule bound thereto.

Preferably, the non-covalent binding group (—B) is not a polynucleotide group.

Preferably, the non-covalent binding group (—B) has molecular weight of 1,000 kDa or less, more preferably 800 kDa or less.

Preferably, the non-covalent binding group (—B) binds to a secondary or tertiary structure within the target nucleic acid, such as a quadruplexes, a pseudoknot, a triplex, a tetraloop, a step-loop or a hairpin loop. More preferably, the non-covalent binding group (—B) binds to a quadruplex or pseudoknot.

Preferably, the bifunctional molecule binds to a secondary or tertiary structure with a dissociation constant (k_{D}) of 10,000 nM or less, such as determined by SPR, or alternatively by microscale thermophoresis (MST) or by a fluorescence quenching assay. Preferably, the bifunctional molecule binds to the secondary or tertiary structure with a k_{D} of 1,000 nM or less, more preferably 500 nM or less, even more preferably 200 nM or less, and most preferably 100 nM or less.

Additionally, or alternatively, the bifunctional molecule binds to a secondary or tertiary structure with a dissociation constant (k_{D}) of 10 mM or less, such as determined by microscale thermophoresis (MST). Preferably, the bifunctional molecule binds to the secondary or tertiary structure with a k_{D} of 8 mM or less, more preferably 7 mM or less, more preferably 6 mM or less, and even more preferably 5 mM or less.

Preferably, the non-covalent binding group (—B) is selected from formulae (B-I) to (B-III) set out below.

Preferably, the target nucleic acid molecule is an RNA molecule, such as viral RNA or a bacterial ribozyme.

Preferably, the target nucleic acid molecule is contacted with the bifunctional molecule within a cell.

In a second aspect of the invention, there is provided a method for identifying a secondary or tertiary structure within a target nucleic acid molecule, the method comprising:
providing first and second populations of nucleic acid molecules, each population comprising the target nucleic acid molecule;
introducing into the first population of nucleic acid molecules a bifunctional molecule of formula (I):

C-L-B (I)
where —C is a cleavage group that is imidazole, optionally substituted with 1 to 3 C₁₋₆ alkyl groups, -L- is a linker and —B is a non-covalent binding group, such that the bifunctional molecule non-covalently binds to the target nucleic acid molecule;
allowing the bifunctional molecule to cleave the target nucleic acid molecule present in the first population; and
identifying nucleic acid molecules which are present in a reduced amount in the first population relative to the second population.

Preferred embodiments of the bifunctional molecule of formula (I) set out for the first aspect also apply to the second aspect.

In a third aspect of the invention, there is provided a bifunctional molecule of formula (I) for use in a method of treatment:

C-L-B (I)
where —C is a cleavage group that is imidazole, optionally substituted with 1 to 3 C₁₋₆alkyl groups, -L- is a linker —B is a non-covalent binding group.

Preferred embodiments of the bifunctional molecule of formula (I) set out for the first aspect also apply to the third aspect.

Preferably, the treatment is treatment of a bacterial or viral infection.

Preferably, the viral infection is an infection with an RNA virus, more preferably a (+)ssRNA virus, even more preferably a coronavirus.

Preferably, the treatment is treatment of a respiratory tract infection, a urinary tract infection, or gastroenteritis.

Additionally, or alternatively, the treatment is treatment of cancer.

In a fourth aspect of the invention, there is provided a bifunctional molecule of formula (I):

C-L-B (I)
where —C is a cleavage group that is imidazole, optionally substituted with 1 to 3 C₁₋₆ alkyl groups, -L- is a linker, and —B is a non-covalent binding group that binds to a nucleic acid molecule.

Preferred embodiments of the bifunctional molecule of formula (I) set out for the first aspect also apply to the fourth aspect.

Preferably, the bifunctional molecule is selected from compounds Deg-I to Deg-V.

These and other aspects and embodiments of the invention are described in further detail below.

### SUMMARY OF THE FIGURES

The present invention is described with reference to the figures listed below.

FIG. 1: Schematic diagram of the mode of action of degraders. FIG. 1 demonstrate the use of the degraders in a coronaviral pseudoknot degradation strategy. The pseudoknot-degrader binds and then directly degrades the coronaviral region that contains the pseudoknot without a need for other agents.

FIG. 2: rG4 degraders cleave rG4-containing oligomers and SARS-CoV-2 genome in vitro. (a) Shows the effect of rG4 degraders on rG4-competent oligomer. Degraders cleave oligomers under conditions that facilitate rG4 formation. n=3. (b) Shows the effect of rG4 degraders on an oligomer that does not form rG4s. No degradation was observed. n=3. (c) Shows nanopore sequencing data indicating widespread degradation on SARS-CoV-2 genome upon treatment with rG4 degrader PDS-deg6 (9A) on its ORF1b. * p<0.05, ** p<0.01, *** p<0.005, n.s. not significant.

FIG. 3: In vitro preliminary findings of G4-degraders anti-SARS-CoV-2 activity. (a) Shows the inhibition of plaque forming units (PFU) on samples treated at 50 μM with PDS-deg4 (9B), PDS-deg6 (9A) and PDS-DegALK (8). (b) Provides PCR measurements of viral RNA. The results show the inhibition of viral replication by PDS-deg6 (9A) at 5 μM and 50 μM. As shown, PDS-Imi6 (9A) appears to inhibit viral growth to a greater extent than PDS-deg4 (9B). (c) Shows the viability of cells after 24 hours of incubation with increasing concentrations of the G4-degrader. None of the compounds showed cytotoxicity up to 50 PM.

FIG. 4: In vivo anti-SARS-CoV-2 activity of G4-degrader. (a) Mice administered with PDS-deg4 (9B) (purple, triangular markers) showed 10% loss of body weight in the first day after infection, which stabilized between Day 1 and Day 3 before decreasing again to reach the 75% threshold on Day 5, as observed in vehicle (0.1% DMSO in water) treated animals (grey, square markers). Non-infected mice treated with vehicle (0.1% DMSO in water) as a control (circular markers) as showed no decrease in body-weight. (b) Quantification of lung viral load on Day 5 by plaque assay showed decreased loads in animals treated with PDS-deg4 (9B) (purple, right) in comparison to vehicle control group (grey, left). * p<0.01.

FIG. 5: MTDB-degrader (16a) cuts coronaviral pseudoknots in vitro. (a) Shows the synthetic design of MTDB-deg (16a). (b) Shows the structure of control molecule TDB-deg (16b) that features a weak pseudoknot binder and the imidazole cleavage portion. (c) Is LC-MS data showing the degradation of the pseudoknot in the presence of the degraders against control, n=3. (d) Shows gel photographs validating the activity of the pseudoknot degraders. (e) Is LCMS data showing that pseudoknot degraders loose efficiency when one of the pseudoknot stems is mutated to perturb the pseudoknot secondary structure, n=3. (f) Are gels photographs showing the degradation of native RNA extracted from SARS-CoV-2 by MTDB-deg (16a) against control. n.s.—not significant.

FIG. 6: Direct RNA Nanopore sequencing reveals the genomic loci which get degraded with MTDB-deg. (a) Shows distribution and abundance of aligned reads flanking the pseudoknot area for control- (0.1% DMSO in water) or MTDB-deg-treated SARS-CoV-2 RNA, based on alignments with minimap2. b, Distribution and abundance of aligned reads mapped exclusively on the S sgRNA region for control- or MTDB-deg-treated SARS-CoV-2 RNA, based on alignments with minimap2

FIG. 7: Treatment with MTDB-deg has no effect on the subgenomic SARS-CoV-2 RNAs. Distribution and abundance of aligned reads mapped exclusively on the indicated sgRNA regions for control- (0.1% DMSO in water) or MTDB-deg-treated SARS-CoV-2 RNA, based on alignments with minimap2.

FIG. 8: MTDB-degrader inhibits SARS-CoV-2 replication in cells. (a), (b) Show the percentage of inhibition of viral replication normalised to vehicle control (dashed line) after incubation with increasing concentrations of the pseudoknot degrader (MTDB-deg (16a)) and control molecules (MTDB and TDB-deg (16b)). Viral replication was assessed after 24 h of infection (multiplicity of infection (MOI) of 0.05) based on E gene and Pseudoknot region RNA levels. Antiviral activity of the MTDB-deg (16a) was observed both before (a), and after infection (b), with SARS-CoV-2 at a 0.05 MOI. Mean ±SD of triplicates is shown and differences between means with p<0.01 are indicated. *, p<0.05; **, p<0.01; two-tailed paired t-tests. (c) Shows that the 50% inhibitory concentration (IC₅₀) of the pseudoknot degrader MTDB-deg (16a) is lower when drug is added after infection. (d) Are photographs of cell monolayers after 4 days of incubation with supernatants from viral cultures that were treated with 6 μM of MTDB-deg (16a), MTDB and TDB-deg (16b) for 24 h. Treatment with the MTDB-deg (16a) at 6 mM for 24 h showed a decreased number of viral plaques in comparison to vehicle control, both when added before or after infection. Control molecule MTDB only showed decreased number of viral plaques when added before infection, and TDB-deg (16b) showed no decrease. (e) Cell viability assay demonstrating that none of the compounds showed cytotoxicity in VeroCCL81 cells after 24 h. (f) Shows the percentage of viral replication relative to vehicle control 24 h after removal of the degrader-containing medium. 24 h treatment with MTDB-deg (16a) 24 h compromised the ability of virus to recover from drug exposure.

FIG. 9: Dose-response curves of MTDB-deg, MTDB and TDB-deg. (a), (b) Show the 50% inhibitory concentration (IC₅₀) values for pseudoknot degrader MTDB-deg (16a) before and after infection. Control molecules (MTDB and TDB-deg (16b) did not inhibit viral replication and thus IC₅₀ values could not be determined. (c) Is a dose-response curve (as determined by PCR targeting the E gene), including a higher concentration of 18 μM showing increased IC₅₀.

FIG. 10: Viral recovery and virucidal activity after MTDB-degrader exposure. (a) Virus ability to recover after 24 h incubation with MTDB-deg (16a) and control molecules MTDB and TDB-deg (16b), as determined by qPCR targeting the pseudoknot region. Viral recovery was impaired in MTDB-deg (16b) treated samples, but not in samples treated with the control molecules MTDB and TDB-deg (16b). (b) Virucidal activity was assessed by incubating 1000 PFU of SARS-CoV-2 with compounds at 6 μM, for 1 h at 37° C. after which residual viral infectivity was determined by plaque assay. MTDB-deg (16a), MTDB and TDB-deg (16b) showed no virucidal effect on cell free virions, suggesting that the MTDB-deg (16a) antiviral activity is mediated by inhibiting virus replication in host cells and not by inactivation of cell free virions.

FIG. 11: Agarose gel analysis of ribosome degradation assay. Left: ethyl linker, no degradation visible. Centre left: diethylene glycol linker, degradation visible at 15 mM concentration. Centre right: hexaethylene glycol linker, no degradation visible. Right: control chloramphenicol, no degradation. B=Blank (no degrader), concentration degrader: 1a/1b=15 mM, 2a/2b=7.5 mM, 3a/3b=3.75 mM, 4a/4b=1.88 mM, 5a/5b=0.94 mM, 6a/6b=0.47 mM.

FIG. 12: MTDB-degrader in vivo activity against SARS-CoV-2 infection in K18-hACE2 mice. (a) Eight- to twelve-week-old female K18-hACE2-transgenic mice were intranasally infected with 104 plaque-forming units (PFU) of SARS-CoV-2 and treated intranasally 1 hour pre-infection and 3 hours post-infection with MTDB-deg 16a (25 mg/kg) (n=6), MTDB (10 mg/kg, maximal dose that could administered given limited solubility) (n=3), TDB-deg 16b (25 mg/kg) (n=5) and vehicle control (n=6). (b) Administration of MTDB-deg 16a leads to a decrease in lung viral load of SARS-CoV-2 infected K18-hACE2 mice. No differences in lung viral load between vehicle control and MTDB and TDB-deg 16b treated mice were observed. Mean ±SD is shown; *p<0.05; unpaired t-test. (c) Western blot analysis of phospho-p38 from lung extracts of transgenic K18-hACE2 mice treated with three doses of 10 mg/kg of vehicle (V1, V2) or MTDB-deg 16a (D1, D2) at 1 hour before infection and 1 and 2 days after infection (n=2).

FIG. 13: RT-qPCR validation of degradation specificity in a cellular system. SARS-CoV-2 infected Vero CCL-81 cells were treated with either 6 μM MTDB-deg or a vehicle (H2O) control for 24 hours (n=3). Student's t test. Means +SD of three independent replicates are shown, * p<0.05, ** p<0.01.

FIG. 14: Binding affinities of PDS and MTDB families of molecules towards their targets. (a) Binding affinities of the PDS family of ligands towards G4-forming NRAS oligonucleotide, as determined via fluorescence quenching assay. (b) Binding affinities of MTDB family of ligands towards pseudoknot oligonucleotide, as measured via MST. (c) Binding affinities of MTDB family of ligands towards perturbed pseudoknot oligonucleotide, as measured via MST. n=3.

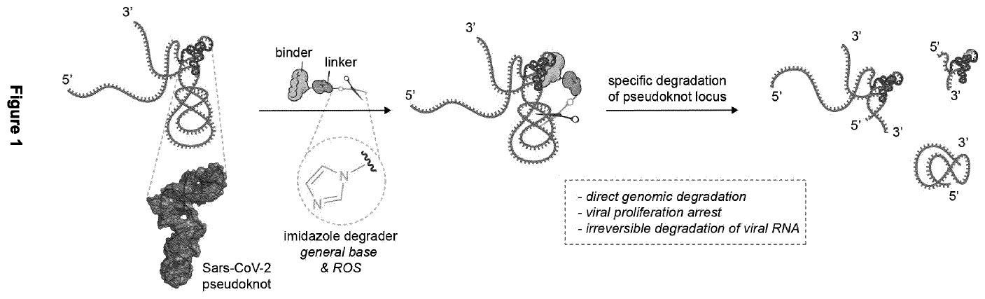

### DETAILED DESCRIPTION OF THE INVENTION

The present invention relates to the finding that a bifunctional molecule, also known herein as a degrader, can be used as a catalytic agent to non-covalently bind to and cleave a target nucleic acid molecule. The degrader disclosed herein bind to a target nucleic acid through non-covalent interactions. Accordingly, the degrader does not require incorporation of a click-reactive group into the target nucleic acid. The selective cleavage of target nucleic acid molecules using the degraders described herein may be useful in epigenetic and epitranscriptomic analysis, bifunctional mapping, as well as in therapy, for example anti-bacterial and anti-viral therapy, and additionally anti-cancer.

The degrader has formula (I):

C-L-B (I)

where C is a cleavage group, -L- is a linker and —B is a non-covalent binding group.

### Cleavage Group (—C)

The cleavage group is imidazole (1,3-diazole), which may optionally be substituted as described herein.

The imidazole cleavage group of the degrader is capable of reacting with a target nucleic acid molecule to cleave the target nucleic acid molecule. Typically, the imidazole is capable of abstracting a proton from the hydroxyl group at the 2′ position of a ribose sugar. Optionally, the imidazole is capable of binding copper to induce copper-mediated RNA degradation (Li, Zhong-Rui, et al. Nat Chem 11.10 (2019): 880-889; Wong, K, et al. Can J Biochem 52.11 (1974): 950-958; Subramaniam, Siddharth, et al. F1000Research 4 (2015)).

Imidazole is a basic group. That is, imidazole is capable of accepting a hydrogen cation (H+). The imidazole is also capable of donating an electron pair.

The basicity of a group may be quantitatively assessed using the pKa of the associated conjugate acid. That is, the basicity of basic group [Ba] may be assessed using the pKa of the conjugate acid [BaH]⁺. The pKa of the conjugate acid may be known or it may be determined using standard techniques, such as acid-base titration. Without wishing to be bound by theory, the inventors believe the basic residues having a conjugate acid with a pKa value above a certain threshold, such as a pKa of 6.5 or greater, for example 6.8 or greater, are capable of deprotonating the hydroxyl group at the 2′ position of a ribose sugar in order to permit cleavage of the phosphodiester backbone within a target nucleic acid. Imidazole has a pKa close to 7.

Imidazole comprises a nitrogen atom having a lone electron pair. Groups comprising a nitrogen atom having a lone electron pair are typically capable of coordinating copper. Imidazole is known to chelate copper.

The imidazole may be unsubstituted, or it may be substituted by one, two or three C₁₋₆ alkyl groups, which may be the same or different.

An alkyl group is monovalent saturated hydrocarbon group. The alkyl group may be a C₁₋₆ alkyl group, for example C₁₋₄ alkyl group. In this context, the prefix (e.g. C₁₋₆) denotes the number of carbon atoms in the hydrocarbon backbone. The alkyl group may be linear or branched.

Examples of C₁₋₆ linear alkyl groups include methyl (-Me), ethyl (-Et), n-propyl (-nPr), n-butyl (-nBu), n-pentyl (-Amyl) and n-hexyl. Examples of C₁₋₆ branched alkyl groups include iso-propyl (-iPr), iso-butyl (-iBu), sec-butyl (-sBu), tert-butyl (-tBu), iso-pentyl, neo-pentyl, iso-hexyl and neo-hexyl.

In some preferred embodiments, the imidazole is selected from the groups represented by formula (C-I) to (C-Ill):

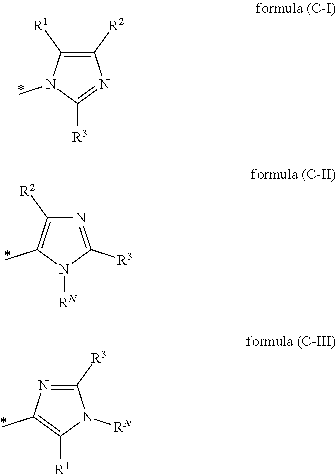

where: R¹, R² and R³ each independently represent a hydrogen atom or a C₁₋₆ alkyl group;
R^{N} represents a hydrogen atom or a C₁₋₆ alkyl group; and
represents the attachment position with the remainder of the molecule (typically the linker unit L).

In such cases, the degrader may be referred to as an imidazole degrader.

Preferably, R¹, R², R³ and R^{N} each independently represent a hydrogen atom or a C₁₋₄ alkyl group.

More preferably, the cleavage group is a group represented by formula (C-I).

Even more preferably, R¹, R², and R³ each independently represent hydrogen. In such case, the cleavage group is an unsubstituted imidazole group. That is, the cleavage group is represented by formula (C-IV):

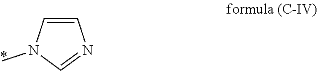

where * represents the attachment position with the remainder of the degrader (typically the linker unit L).

When non-covalently bound to the target nucleic acid molecule through the linker and binding group, the imidazole group reacts with the target nucleic acid molecule to cleave one or more phosphodiester bonds, thereby causing degradation of the target nucleic acid molecule. For example, the imidazole group of the bound degrader may abstract a proton from the 2′OH position on the nucleic acid molecule leading to cleavage of a phosphodiester bond in the target nucleic acid molecule. In addition, the imidazole group may form a copper complex which cleaves a phosphodiester bond in the target nucleic acid molecule.

### Linker (-L-)

The linker L of the degrader comprises a group for connection (i.e. covalent connection) of the cleavage group (C) to the non-covalent binding group (B). Suitable linkers are well known in the art.

Typically, the linker comprises a divalent group in which one of the free valencies forms part of a single bond to the cleavage group (C) and the remaining free valency forms part of a single bond to the non-covalent binding group (B).

Preferably, the linker is a stable linker. That is, the linker comprises a group that is not substantially cleaved or degraded in vivo. A stable linker is typically unreactive at physiological pH, and not substantially degraded by enzymatic action in vivo.

Typically, the linker is a flexible linker. That is, the linker permits the cleavage group (C) and binding group (B) to move relative to each other with a large degree of freedom.

Typical linkers comprise groups selected from alkylene, heteroalkylene, cycloalkylene, heterocycloalkylene, arylene and heteroarylene. Mixed linkers comprising different groups in covalent connection, such as alkylene-arylene (aralkylene) and heteroalkylene-arylene, may be permitted.

An alkylene (alkanediyl) group is a divalent saturated hydrocarbon group in which the two free valencies each form part of a single bond to an adjacent atom. The alkylene group may be a C₁₋₆ alkylene group, for example, a C₁₋₄, C₁₋₃ or a C₁₋₂ alkylene group. In this context, the prefix (e.g. C₁₋₆) denotes the number of atoms in the hydrocarbon backbone. The alkylene group may be linear or branched. Examples of linear alkylene groups include methanediyl (methylene bridge), ethane-1,2-diyl (ethylene bridge), propane-1,3-diyl, butan-1,4-diyl, pentan-1,5-diyl and hexan-1,6-diyl. Examples of branched alkylene groups include ethane-1,1-diyl and propane-i1,2-diyl.

A heteroalkylene group is an alkylene group in which one or more carbon atoms is replaced with a heteroatom, for example N, O and S. The heteroalkylene group may be a C₁₋₆ heteroalkylene group, for example, a C₁₋₄, C₁₋₃ or a C₁₋₂ heteroalkylene group. In this context, the prefix (e.g. C₁₋₆) denotes the number of atoms in the heteroalkylene backbone, whether carbon atoms or heteroatoms. The heteroalkylene group may be linear or branched.

Examples of linear heteroalkylene groups include those derived from oxymethylene (e.g. polyoxymethylene, POM), ethylene glycol (e.g. polyethylene glycol, PEG), ethylenimine (e.g. linear polyethylenimine, PEI; polyaziridine) and tetramethylene glycol (e.g. polytetramethylene glycol, PTMEG; polytetrahydrofuran). Examples of branched heteroalkylene groups include those derived from propylene glycol (e.g. polypropylene glycol PPG). Where a nitrogen atom is present in a heteroalkylene group, that nitrogen atom may be unsubstituted (NH) or optionally substituted with an alkyl group, such as a C₁₋₄ alkyl group. Where a sulfur atom is present in a heteroalkyl group, that sulfur atom may be S, S(O) or S(O)₂.

A cycloalkylene group is a divalent saturated hydrocarbon group which comprises a ring in which all of the ring atoms are carbon atoms, and in which the two free valencies each form part of a single bond to an adjacent atom. The cycloalkylene group may be a C₅₋₆ cycloalkylene group. In this context, the prefix (e.g. C₅₋₆) denotes the number or range of ring atoms. The cycloalkylene group may be monocyclic. Examples of monocylic cycloalkylene groups include 1,3-cyclopentylene and 1,4-cyclohexylene.

A heterocycloalkylene (heterocyclene) group is a cycloalkylene group in which one or more carbon atoms is replaced with a heteroatom, for example N, O and S, or in which one or more carbon atoms has an oxo substituent (═O). The heterocycloalkylene group may be a C₅₋₆ heterocycloalkylene group. In this context, the prefix (e.g. C₅-6) denotes the number or range of ring atoms, whether carbon atoms or heteroatoms. The heterocycloalkylene group may be monocyclic. Where a nitrogen atom is present in a heteroalkylene group, that nitrogen atom may be unsubstituted (NH) or optionally substituted with an alkyl group, such as a C₁₋₄ alkyl group. Where a sulfur atom is present in a heteroalkyl group, that sulfur atom may be S, S(O) or S(O)₂.

An arylene group is a divalent hydrocarbon group comprising an aromatic ring in which all of the ring atoms are carbon atoms, and in which the two free valencies each form part of a single bond to an adjacent atom. The arylene group may be a C₆₋₁₀ arylene group. In this context, the prefix (e.g. C₆₋₁₀) denotes the number or range of ring atoms. The arylene group may be monocyclic, or it may comprise two or more rings. Examples of monocyclic arylene groups include 1,4-phenylene. Examples of bicyclic arylene groups include 2,6-naphthylene.

A heteroarylene group is an arylene group comprising an aromatic ring in which one or more ring atoms are heteroatoms, for example N, O and S, or in which one or more carbon atoms has an oxo substituent (═O). The heteroarylene group may be a C₆₋₁₀ heteroarylene group.

In this context, the prefix (e.g. C₆₋₁₀) denotes the number or range of ring atoms, whether carbon or heteroatom. The heteroarylene group may be monocyclic, or it may comprise two or more rings. Examples of monocyclic heteroarylene groups include pyrrolylene and pyridylene.

Preferred linkers comprise groups selected from alkylene and heteroalkylene. More preferred linkers comprise heteroalkylene groups. Even more preferred linkers comprise alkylene ether groups. Most preferred linkers comprise ethylene oxide groups (e.g. derived from polyethylene glycol, PEG).

In a preferred embodiment, the linker is or comprises a group represented by formula (L-1):

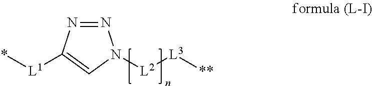

where:
   L¹ is a covalent bond or a C₁₋₂ alkylene group;
   L² is a C₁₋₆ alkylene group or a C₁₋₆ heteroalkene group;
   L³ is a C₁₋₆ alkylene group;
   n is 1 to 8;
   * is the attachment point with the non-covalent binding group (—B); and
   ** is the attachment point with the cleavage group (—C).

Suitable C₁₋₂ alkylene groups include methylene (methanediyl), ethylene (ethane-1,2-diyl).

Suitable C₁₋₆ alkylene groups include methylene (methanediyl), ethylene (ethane-1,2-diyl), propylene (propane-1,3-diyl), butylene (butan-1,4-diyl), pentylene (pentan-1,5-diyl) and hexylene (hexan-1,6-diyl).

Preferably L¹ is a covalent bond or methylene.

Preferably L³ is C₁₋₄ alkylene. More preferably L³ is ethylene.

Preferably n is 2 to 5.

Suitable C₁₋₆ heteroalkene groups include alkylene ether group such as ethylene oxide (—CH₂CH₂O—), propylene oxide (—CH₂CH₂CH₂O—) and tetramethylene oxide (—CH₂CH₂CH₂CH₂O—).

Preferably L² is ethylene oxide. In such cases, the linker is or comprises a group represented by formula (L-II):

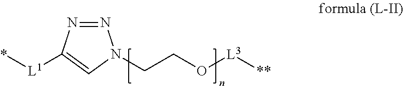

where L¹, L³, n, * and ** are as described for formula (L-I), and the same preferences apply.

In an alternative embodiment, the linker is or comprises a group represented by formula (L-III):

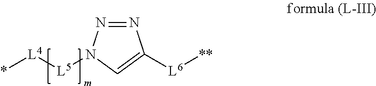

where:
   L⁴ is a C₁ 6 alkylene group;
   L⁵ is a C₁ 6 alkylene group or a C₁₋₆ heteroalkene group;
   L⁶ is a covalent bond or a C₁₋₂ alkylene group;
   m is 1 to 8;
   * is the attachment point with the non-covalent binding group (—B); and
   ** is the attachment point with the cleavage group (—C).

Preferably L⁴ is C_{1 4} alkylene. More preferably L³ is ethylene.

Preferably L⁶ is methylene or ethylene.

Preferably m is 2 to 5.

Preferably L⁵ is ethylene oxide. In such cases, the linker is or comprises a group represented by formula (L-IV):

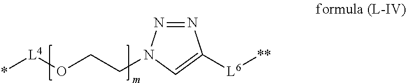

where L⁴, L⁶, m, * and ** are as described for formula (L-III), and the same preferences apply.

### Non-Covalent Binding Group (—B)

The binding group of the degrader comprises a group capable of binding to the target nucleic acid molecule. The binding group binds to the target nucleic acid molecule through non-covalent bonding.

Certain small-molecule ligands including are known to non-covalently bind to nucleic acids, and thus may form the basis of the non-covalent binding group.

The non-covalent binding group is not a polynucleotide (e.g. nucleic acid) group. The covalent binding group is not or does not comprise a nucleotide.

The non-covalent binding group is not an antibody.

Typically, the non-covalent binding group has molecular weight of 1,000 kDa or less. Preferably, the non-covalent binding group has a molecular weight of 800 kDa or less.

Typically, the non-covalent binding group binds to a secondary or tertiary structure within the target nucleic acid. Suitable secondary or tertiary structures include quadruplexes, pseudoknots, triplexes, tetraloops, step-loops and hairpin loops. Preferably, the non-covalent binding group binds to a quadruplex or pseudoknot.

Preferably, the non-covalent binding group selectively binds to a secondary or tertiary structure within the target nucleic acid. In such cases, the non-covalent binding group preferentially binds to a secondary or tertiary structure within the target nucleic acid in comparison to linear or unstructured nucleic acid. Preferably, the non-covalent binding group selectively binds to a quadruplex or pseudoknot.

Preferably, the non-covalent binding group selectively binds to a ribonucleic acid (RNA). Accordingly, the non-covalent binding group may be known as a non-covalent RNA binding group.

The non-covalent binding group may bind to the target nucleic acid through electrostatic interactions, such as ionic interactions, hydrogen-bonding and halogen bonding; van der Waals interactions such as permanent dipole-dipole interactions, dipole-induced dipole interactions, and induced dipole-induced dipole interactions; and π-effects such as π-π interactions, π-cation interactions and polar-π interactions.

The non-covalent binding group may be based on the following small molecule nucleic acid binding molecules:

| Name | Structure | Binding group |
|---|---|---|
| Pyridostatin (PDS) | 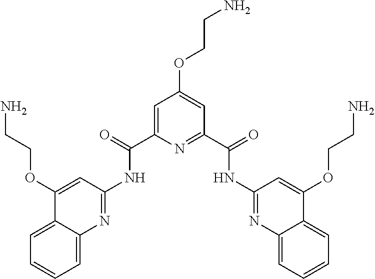 | 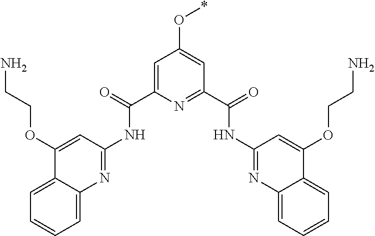 [SMILES: *Oc1cc(C(=O)Nc2cc(OCCN)c3ccccc3n2)nc(C(=O)Nc2cc(OCCN)c3ccccc3n2)c1] |
| | | (B-I) |
| MTBD | 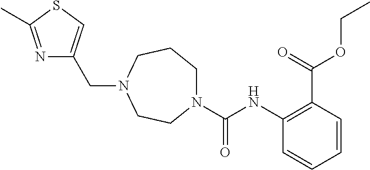 | 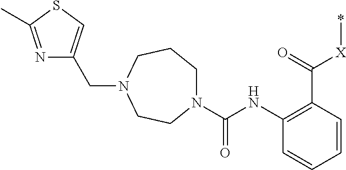 |
| | | X is O or NH |
| | | (B-II) |
| Chloramphenicol | 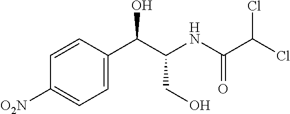 | 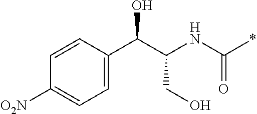 [SMILES: *C(=O)N[C@H](CO)[C@H](O)c1ccc([N+](=O)[O-])cc1] |
| | | (B-III) |
| | | *is the attachment point with the |
| | | linker |

The non-covalent binding group may be attached to the linker at any suitable position. Typically, the non-covalent binding group is attached to the linker through a heteroatom (such as O or NH), or adjacent to a carbonyl group (C═O).

Preferably, the binding group is selected from formulae (B-I) to (B-III).

### Kinetic Properties

The interaction between the degrader and the target nucleic acid can be quantified using the dissociation constant (k_{D}). The dissociation constant between a degrader comprising a given non-covalent binding group and a nucleic acid may be known or it may be determined using standard techniques such as surface plasmon resonance (SPR), for example Biacore (Santos, et al., 2021). Suitable systems for measuring the dissociation constant include Biacore T200.

Typically, the degrader binds to the target nucleic acid with a dissociation constant (k_{D}) of 10,000 nM or less, such as determined by SPR. Preferably, the degrader binds to the target nucleic acid with a k_{D} of 1,000 nM or less, more preferably 500 nM or less, even more preferably 200 nM or less, and most preferably 100 nM or less.

As noted above, the non-covalent binding group of degrader typically binds to a secondary or tertiary structure within the target nucleic acid. Accordingly, the degrader typically binds to a secondary or tertiary structure with a dissociation constant (k_{D}) of 10,000 nM or less, such as determined by SPR. Preferably, the degrader binds to the secondary or tertiary structure with a k_{D} of 1,000 nM or less, more preferably 500 nM or less, even more preferably 200 nM or less, and most preferably 100 nM or less.

In some embodiments, the degrader binds to a quadruplex with a dissociation constant (k_{D}) of 10,000 nM or less, such as determined by SPR. In such cases, the degrader preferably binds to the quadruplex with a k_{D} of 1,000 nM or less, more preferably 500 nM or less, even more preferably 200 nM or less, and most preferably 100 nM or less.

In some embodiments, the degrader binds to a pseudoknot with a dissociation constant (k_{D}) of 10,000 nM or less, such as determined by SPR. In such cases, the degrader preferably binds to the pseudoknot with a k_{D} of 1,000 nM or less, more preferably 500 nM or less, even more preferably 200 nM or less, and most preferably 100 nM or less.

In additional or alternative embodiments, the degrader binds to the target nucleic acid with a dissociation constant (k_{D}) of 100 mM or less, such as determined by microscale thermophoresis (MST). In such cases, the k_{D} may be 50 mM or less, such as 25 mM or less, such as 20 mM or less, such as 15 mM or less, such as 10 mM or less, such as 9 mM or less, such as 8 mM or less, such as 7 mM or less, such as 6 mM or less, such as 5 mM or less, such as 4 mM or less, such as 3 mM or less, such as 2 mM or less. Preferably, the degrader binds to the target nucleic acid with a dissociation constant (k_{D}) of 10 mM or less, more preferably 5 mM or less.

The k_{D} may be determined using standard techniques such as by microscale thermophoresis (MST), such as described in the Examples below. Measurements may be carried out using a fluorescence-tagged nucleic acid, such as FAM-functionalised nucleic acid which is incubated with the degrader. The nucleic acid may be at a concentration of 50 nM, and may be provided in a buffer such as a HEPES buffer at pH 7.4. The degrader may be tested in serial dilution, such as at a highest concentration of up to 8 mM, such as up to 250 PM.

Measurements may be made at a temperature of 25° C. MST measurements may be carried out at an MST power of 30%. Suitable systems for measuring the dissociation constant include NanoTemper Monolith NT.115.

Preferably, the degrader binds to a secondary or tertiary structure within the target nucleic acid with a k_{D} of 20 nM or less, more preferably 15 nM or less, more preferably 10 mM or less, more preferably 5 mM or less.

In some embodiments, the degrader binds to a pseudoknot with a dissociation constant (k_{D}) of 20 mM or less, more preferably 15 mM or less, more preferably 10 mM or less, even more preferably 5 mM or less, and most preferably 2 mM or less.

Additionally, or alternatively, the interaction between the degrader and the target nucleic acid can be quantified using the half maximal effective constant (EC₅₀). The EC₅₀ may be the same as the dissociation constant (k_{D}) or the EC₅₀ may be different. Preference for the EC₅₀ is as described above for the dissociation constant (k_{D}).

The half maximal effective constant (EC₅₀) between a degrader comprising a given non-covalent binding group and a nucleic acid may be known, or it may be determined experimentally, for example using a fluorescence quenching assay (see e.g.

Di Antonio, et al., 2012 and the methods described in the Examples below). Suitable systems for measuring the EC₅₀ include systems for measuring fluorescence, such as a plate reader, such as BMG CLARIOstar. The nucleic acid may be a fluorescence-tagged nucleic acid which may be treated with the degrader for a time period such as 40 minutes, such as with incubation at a temperature of 4° C. Measurements may be carried out at a temperature of 25° C. The concentration of the nucleic acid may be 50 nM, and optionally the nucleic acid may be in a buffer such as a HEPES buffer at pH 7.4. The concentration of the degrader may be up to 10 μM, for example where the degrader is tested in serial dilution from around 2 nM to around 10 μM.

Typically, the degrader binds to the target nucleic acid with a half maximal effective constant (EC₅₀) of 10,000 nM or less, such as determined by a fluorescence quenching assay.

Preferably, the degrader binds to the target nucleic acid with an EC₅₀ of 1,000 nM or less, more preferably 500 nM or less, even more preferably 200 nM or less, and most preferably 100 nM or less.

As noted above, the non-covalent binding group of degrader typically binds to a secondary or tertiary structure within the target nucleic acid. Accordingly, the degrader typically binds to a secondary or tertiary structure with a half maximal effective constant (EC₅₀) of 10,000 nM or less. Preferably, the degrader binds to the secondary or tertiary structure with an EC₅₀ of 1,000 nM or less, more preferably 500 nM or less, even more preferably 200 nM or less, and most preferably 100 nM or less.

In some embodiments, the degrader binds to a quadruplex with a half maximal effective constant (EC₅₀) of 10,000 nM or less, such as determined by a fluorescence quenching assay as described herein. In such cases, the degrader preferably binds to the quadruplex with a EC₅₀ of 1,000 nM or less, more preferably 500 nM or less, even more preferably 200 nM or less, and most preferably 100 nM or less.

In some embodiments, the degrader binds to a pseudoknot with a half maximal effective constant (EC₅₀) of 10,000 nM or less, such as determined by a fluorescence quenching assay as described herein. In such cases, the degrader preferably binds to the pseudoknot with a EC₅₀ of 1,000 nM or less, more preferably 500 nM or less, even more preferably 200 nM or less, and most preferably 100 nM or less.

In some embodiments, the degrader binds to the target nucleic acid with a half maximal effective constant (EC₅₀) of 100 mM or less, such as 50 mM or less, such as 25 mM or less, such as 20 mM or less, such as 15 mM or less, such as 10 mM or less, such as 9 mM or less, such as 8 mM or less, such as 7 mM or less, such as 6 mM or less, such as 5 mM or less, such as 4 mM or less, such as 3 mM or less, such as 2 mM or less. Preferably, the degrader binds to the target nucleic acid with a half maximal effective constant (EC₅₀) of 10 mM or less, more preferably 5 mM or less.

Preferably, the degrader binds to a secondary or tertiary structure within the target nucleic acid with a half maximal effective constant (EC₅₀) of 20 nM or less, more preferably 15 nM or less, more preferably 10 mM or less, more preferably 5 mM or less.

In some embodiments, the degrader binds to a pseudoknot with a half maximal effective constant (EC₅₀) of 20 mM or less, such as 15 mM or less, more preferably 10 mM or less, even more preferably 5 mM or less, and most preferably 2 mM or less.

As noted above, the non-covalent binding group of the degrader preferably selectively binds to a secondary or tertiary structure within the target nucleic acid. The binding selectivity can be quantified using the ratio between the dissociation constant for binding to a given secondary or tertiary structure in compression to the dissociation constant for binding to linear or unstructured nucleic acid, such as linear or unstructured RNA. Typically, the comparison linear or unstructured nucleic acid is prepared by mutating one or more residues within the secondary or tertiary structure of interest such that the secondary or tertiary structure no longer forms, while the remainder of the sequence is maintained. For example, the selectivity of binding to an RNA G quadruplex can be assessed by using comparison RNA in which one or more GGG motifs are exchanged for AUC motifs.

Typically, the binding selectivity between a given secondary or tertiary structure and linear or unstructured nucleic acid is 5:1 or greater. Preferably, selectivity between a given secondary or tertiary structure and linear or unstructured nucleic acid is 10:1 or greater, more preferably 20:1 or greater, even more preferably 50:1 or greater, and most preferably 100:1 or greater.

In one embodiment, the binding selectivity between a quadruplex and linear or unstructured nucleic acid is 5:1 or greater. Preferably, selectivity between a quadruplex and linear or unstructured nucleic acid is 10:1 or greater, more preferably 20:1 or greater, even more preferably 50:1 or greater nM or less, and most preferably 100:1 or greater.

In one embodiment, the binding selectivity between a quadruplex and linear or unstructured nucleic acid is 5:1 or greater. Preferably, selectivity between a quadruplex and linear or unstructured nucleic acid is 10:1 or greater, more preferably 20:1 or greater, even more preferably 50:1 or greater nM or less, and most preferably 100:1 or greater.

### Preferred Embodiments

In a preferred embodiment, the degrader is selected from compounds Deg-I to Deg-V.

| Compound | Structure |
|---|---|
| Deg-I (PDS-deg6) | 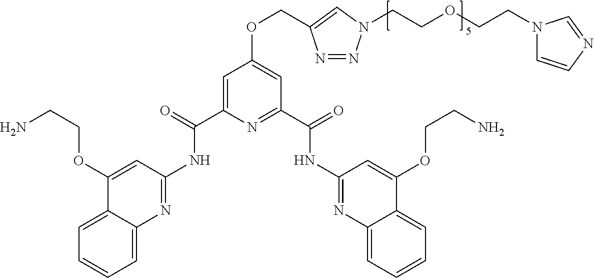 |
| Deg-II (PDS-deg4) | 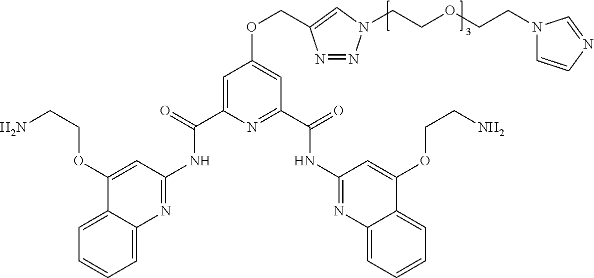 |
| Deg-III MTBD-deg | 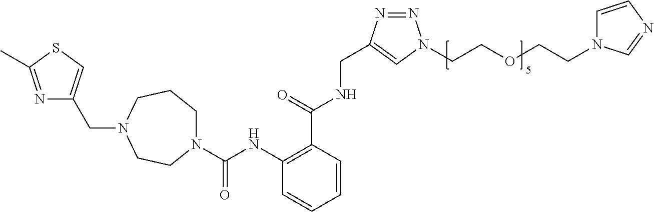 |
| Deg-IV (chlo-deg6) | 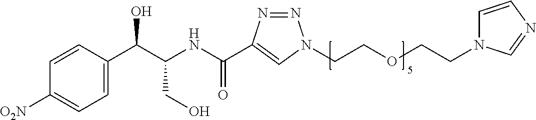 |
| Deg-V (chlo-deg1) | 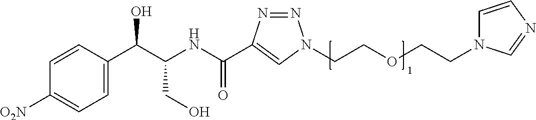 |

### Salts and Solvates

The degrader of formula (I) may be provided in free base form.

The degrader of formula (I) may be provided in the form of a salt, preferably a pharmaceutically acceptable salt.

In some embodiments, degraders disclosed herein may be provided as salts in a protonated form together with a suitable counter anion.

Suitable counterions include both organic and inorganic anions. Example of inorganic anions include those derived from inorganic acids, including chloride (Cl), bromide (Br), iodide (I), sulfate (SO₄₂), sulfite (SO₃₂), nitrate (NO₃), nitrite (NO₂), phosphate (PO₄₃), and phosphite (PO₃₃). Examples of organic anions include 2-acetoxybenzoate, acetate, ascorbate, aspartate, benzoate, camphorsulfonate, cinnamate, citrate, edetate, ethanedisulfonate, ethanesulfonate, formate, fumarate, gluconate, glutamate, glycolate, hydroxymalate, carboxylate, lactate, laurate, lactate, maleate, malate, methanesulfonate, oleate, oxalate, palmitate, phenylacetate, phenylsulfonate, propionate, pyruvate, salicylate, stearate, succinate, sulfanilate, tartarate, toluenesulfonate, and valerate. Examples of suitable polymeric organic anions include those derived from tannic acid and carboxymethyl cellulose.

In some embodiments, degraders disclosed herein may be provided as salts in a deprotonated form together with a suitable counter cation.

Suitable counterions include both inorganic and organic cations. Examples of suitable inorganic cations include alkali metal ions such as Na⁺ and K⁺, alkaline earth cations such as Ca²⁺ and Mg²⁺, and other cations such as Al³⁺. Examples of suitable organic cations include the ammonium ion (i.e., NH₄⁺) and substituted ammonium ions (e.g., NH₃R⁺, NH₂R₂⁺, NHR₃⁺, NR₄⁺). Examples of substituted ammonium ions include those derived from ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. Additional or alternative examples of substituted ammonium ions include those derived from putrescine and spermidine, or polyvalent amines, such as tetramethylethylenediamine (TEMED). An example of a common quaternary ammonium ion is N(CH₃)₄⁺.

The degrader of formula (I) may be provided in the form of a solvate (a complex of solute (e.g., compound, salt of compound) and solvent). Examples of solvates include hydrates, for example, a mono-hydrate, a di-hydrate and a tri-hydrate.

The degrader of formula (I) may be provided in desolvated form, for example, in dehydrated form.

### Methods for Cleaving at Target Nucleic Acid

The invention provides a method for cleaving a target nucleic acid molecule. The method comprises:
contacting the target nucleic acid molecule with a degrader of formula (I):

C-L-B (I)
where —C is a cleavage group that is imidazole, optionally substituted with 1 to 3 C₁₋₆ alkyl groups, -L- is a linker and —B is a non-covalent binding group, such that the degrader non-covalently binds to the target nucleic acid molecule, and;
allowing the degrader to cleave the target nucleic acid molecule bound thereto.

Preferred embodiment of the degrader of formula (I) are set out above.

In some embodiments, the target nucleic acid molecule may be contacted with the degrader in solution.

More preferably, the target nucleic acid molecule may be contacted with the degrader within a cell (i.e. intracellularly). The cell may be in vitro and may be an isolated cell, for example an isolated cell line or cell isolated from an individual (from a tissue sample, such as a biopsy).

Suitable cells may include mammalian, preferably human cells. Cells may include somatic and germ-line cells and may be at any stage of development, including fully or partially differentiated cells or non-differentiated or pluripotent cells, including stem cells, such as adult or somatic stem cells, foetal stem cells or embryonic stem cells. For example, cells may include neural cells, including neurons and glial cells, contractile muscle cells, smooth muscle cells, liver cells, hormone synthesising cells, sebaceous cells, pancreatic islet cells, adrenal cortex cells, fibroblasts, keratinocytes, endothelial and urothelial cells, osteocytes, and chondrocytes. In some embodiments, cells may be associated with a disease condition, for example cancer cells, such as carcinoma, sarcoma, lymphoma, blastoma or germ-line tumour cells, and cells with the genotype of a genetic disorder, such as Huntington's disease, cystic fibrosis, sickle cell disease, phenylketonuria, Down syndrome or Marfan syndrome.

The target nucleic acid molecule may be an endogenous nucleic acid that is present in the cell. The degrader may be an exogenous molecule. A method may comprise introducing the degrader into the cell and allowing it to bind to the target nucleic acid molecule.

The target nucleic acid molecule may be a DNA or RNA molecule. Suitable target RNA molecules may include mRNA and long non-coding RNA (lncRNA). The RNA molecule may comprise intronic and intergenic regions.

The target nucleic acid molecule may comprise a secondary or tertiary structure. Suitable secondary and tertiary structures include quadruplexes, pseudoknots, tetraloops, step-loops and hairpin loops. Preferably, the target nucleic acid molecule comprises a quadruplex or pseudoknot.

For example, a method for cleaving a target nucleic acid comprising a secondary or tertiary structure may comprise:
contacting the target nucleic acid molecule with a degrader of formula (I):

C-L-B (I)
where —C is a cleavage group that is imidazole, optionally substituted with 1 to 3 C₁₋₆ alkyl groups, -L- is a linker and —B is a non-covalent binding group that interacts with the secondary or tertiary structure to non-covalently bind the degrader to the target nucleic acid molecule, and;
allowing the degrader to cleave the target nucleic acid molecule bound thereto.

In a preferred embodiment, the secondary or tertiary structure is a quadruplex. In such cases, the method may comprise:
contacting a target nucleic acid molecule comprising a quadruplex with a degrader of formula (I):

C-L-B (I)
where —C is a cleavage group that is imidazole, optionally substituted with 1 to 3 C₁₋₆ alkyl groups, -L- is a linker and —B is a non-covalent binding group that interacts with the quadruplex to non-covalently bind the degrader to the target nucleic acid molecule, and;
allowing the degrader to cleave the target nucleic acid molecule bound thereto.

In a preferred embodiment, the secondary or tertiary structure is a pseudoknot. In such cases, the method may comprise:
contacting a target nucleic acid molecule comprising a pseudoknot with a degrader of formula (I):

C-L-B (I)
where —C is a cleavage group that is imidazole, optionally substituted with 1 to 3 C₁₋₆ alkyl groups, -L- is a linker and —B is a non-covalent binding group that interacts with the pseudoknot to non-covalently bind the degrader to the target nucleic acid molecule, and;
allowing the degrader to cleave the target nucleic acid molecule bound thereto.

When non-covalently bound to the target nucleic acid molecule, the degrader cleaves the target nucleic acid. Typically, the degrader cleaves one or more phosphodiester bonds in the target nucleic acid.

When non-covalently bound to the target nucleic acid molecule, the cleavage group may abstract a protein from the 2′OH of a nucleotide in the target nucleic acid. Cleavage of the phosphodiester backbone may occur by intramolecular attack on the phosphate group at the 3′ position.

When non-covalently bound to the target nucleic acid molecule, the cleavage may bind to one or more transition metals (e.g. copper). The degrader may cleave the target nucleic acid through copper-mediated nucleic acid degradation.

Binding of the degrader to the target nucleic acid may proceed via an intermediate species. That is, a target nucleic acid molecule may be cleaved as described herein by a method that comprises binding the target nucleic acid molecule to a degrader to produce an intermediate having the formula:

C-L-B˜NA
where —C is a cleavage group that is imidazole, optionally substituted with 1 to 3 C₁₋₆alkyl groups, -L- is a linker, —B is a non-covalent binding group, ˜ is a non-covalent interaction and NA is the target nucleic acid; and
allowing the degrader to cleave the target nucleic acid molecule.

Preferred embodiment of the degrader of formula (I) are set out above.

### Methods for Identifying Secondary or Tertiary Structures

Following selective cleavage of a target nucleic acid molecule by a degrader as described above, a method may comprise identifying the target nucleic acid molecule. This may be useful for example in the mapping of sites comprising secondary or tertiary structures within the nucleic acid.

The method may also comprise determining the abundance or amount of one or more nucleic acid molecules in a population of nucleic acids. A reduction in the abundance or amount of a nucleic acid molecule in the population relative to control is indicative that the nucleic acid molecule is the target nucleic acid molecule that has been selectively cleaved by the degrader. A suitable control may be a population of nucleic acids that has not been treated with the degrader.

Accordingly, the invention provides a method for identifying a secondary or tertiary structure within a target nucleic acid molecule, the method comprising:
providing first and second populations of nucleic acid molecules, each population comprising the target nucleic acid molecule;
introducing into the first population of nucleic acid molecules a degrader of formula (I):

C-L-B (I)
where —C is a cleavage group that is imidazole, optionally substituted with 1 to 3 C₁₋₆ alkyl groups, -L- is a linker and —B is a non-covalent binding group, such that the degrader non-covalently binds to the target nucleic acid molecule;
allowing the degrader to cleave the target nucleic acid molecule present in the first population; and
identifying nucleic acid molecules which are present in a reduced amount in the first population relative to the second population.

Preferred embodiment of the degrader of formula (I) are set out above.

The non-covalent binding group may bind to a secondary or tertiary structure within the target nucleic acid molecule. Suitable secondary or tertiary structures include quadruplexes, pseudoknots, tetraloops, step-loops and hairpin loops. Preferably, the secondary or tertiary structure is a quadruplex or pseudoknot.

The first and second populations of nucleic acid molecules may independently be isolated (ex vivo) populations of nucleic acid molecules. Alternatively, one or more of the populations of nucleic acid molecules may be present within a cell.

The method may comprise extracting the total nucleic acid, such as total DNA or total RNA, from a cell. The nucleic acid may be further analysed, for example to determine the abundance or amount of one or more nucleic acid molecules. For example, the extracted total nucleic acid may be sequenced, and the sequence reads analysed.

Suitable methods of determining the abundance or amount of nucleic acid molecules in a cell are well known in the art and include RT-qPCR, RNA-sequencing (RNA-seq), next generation (NGS), nanopore sequencing, and other sequencing techniques, such as Sanger sequencing, Tracking Indels by Composition (TIDE) (Brinkman et al Nucleic Acids Res. 2014 Dec. 16; 42(22): e168) and PCR analysis. In some embodiments, a method may comprise extracting nucleic acid molecules from the cell, sequencing the extracted nucleic acid molecules and determining the number of sequence reads (i.e. read count) for each extracted nucleic molecule to determine the abundance or amount of each nucleic acid molecule in the cell. In some embodiments, the raw read count may be normalised and expressed in RPKM (reads per kilobase of exon model per million reads) or FPKM (fragments per kilobase of exon model per million reads mapped). Suitable methods of sequencing and sequence analysis are well established in the art.

### Use in Medicine

The selective cleavage of a target nucleic acid molecules by the degrader described above may alter downstream effects of the target nucleic acid molecule. This may be useful, for example, in the treatment or prophylaxis of a disease mediated by the target nucleic acid molecule.

Accordingly, the present invention provides a degrader of formula (I) for use in a method of treatment of the human or animal body by therapy, for example, for use in a method of treatment of a disorder (e.g., a disease).

Another aspect of the present invention pertains to a method of treatment, for example, a method of treatment of a disorder (e.g., a disease), comprising administering a therapeutically-effective amount of a degrader of formula (I) to a subject in need of treatment.

Another aspect of the present invention pertains to use of degrader of formula (I) in the manufacture of a medicament for use in treating a disorder (e.g., a disease). Typically, the medicament comprises the degrader of formula (I).

### Disorders Treated

In one embodiment (e.g., of use in methods of therapy, of use in the manufacture of medicaments, of methods of treatment), the treatment is treatment of a bacterial or viral infection.

Preferably, the viral infection is an infection with an RNA virus (e.g., a virus in which the viral genome comprises single- or double-stranded RNA). Many pathogenic viruses exploit a −1 ribosomal frameshifting as a mechanism for correct translation of proteins and this phenomenon is enabled by secondary RNA structures such as stem-loops and pseudoknots.

Accordingly, targeting these secondary RNA structures with a degrader of formula (I) can cleave and inactivate the viral RNA, and treat the viral infection.

Examples of RNA viruses include (+)ssRNA viruses such as coronaviruses, picornaviruses and togaviruses; (−)ssRNA viruses such as orthomyxoviruses and rhabdoviruses; and dsRNA viruses such as reoviruses.

Preferably, the virus is a (+)ssRNA virus, more preferably a coronavirus. Examples of coronaviruses include alphacoronaviruses such as transmissible gastroenteritis virus, feline coronavirus, canine coronavirus; betacoronaviruses such as middle east respiratory syndrome-related coronavirus (MERS-CoV), murine coronavirus (M-CoV) and severe acute respiratory syndrome-related coronavirus (SARS-CoV, SARS-CoV-2); gammacoronavirus such as avian coronavirus; and deltacoronavirus such as bulbul coronavirus HKU11 and porcine coronavirus HKU15.

The bacterial infection may be an infection with a Gram-negative or Gram-positive bacterium. Both classes of bacteria contain a bacterial ribosome, which is a riboenzyme comprising both protein and RNA units. Accordingly, targeting the RNA units with a degrader of formula (I) can cleave and inactive the bacterial ribosome, and treat the bacterial infection.

Examples of medically-relevant Gram-negative bacteria include Haemophilus influenzae, Klebsiella pneumoniae, Legionella pneumophila and Pseudomonas aeruginosa (which are primarily associated with respiratory problems); Escherichia coliand Enterobacter cloacae (which are primarily associated with urinary problems); and Helicobacter pylori and Salmonella enterica (which are primarily associated with gastrointestinal problems), Neisseria meningitidis (which is primarily associated with meningitis)

Accordingly, in one embodiment, the Gram-negative bacterial species is selected from the group consisting of E. coli, E. cloacae, H. pylori, S. enterica, H. influenzae, K. pneumoniae, L. pneumoniae, L. pneumophila, P. aeruginosa and N. meningitidis.

Examples of medically-relevant Gram-positive bacteria include actinomyces, bacillus, clostridium, corynebacterium (e.g. Corynebacterium diphtheriae), enterococcus, erysipelothrix, listerial (e.g. Listeria monocytogenes), nocardia, staphylococcal, and streptococcal (e.g. Staphylococcus aureus).

Accordingly, in one embodiment, the Gram-negative bacterial genus is selected from the group consisting of actinomyces, bacillus, clostridium, corynebacterium, enterococcus, erysipelothrix, listerial nocardia, staphylococcal, and streptococcal.

In one embodiment (e.g., of use in methods of therapy, of use in the manufacture of medicaments, of methods of treatment), the treatment is treatment of a respiratory tract infection, a urinary tract infection, or gastroenteritis.

In some additional or alternative embodiments (e.g., of use in methods of therapy, of use in the manufacture of medicaments, of methods of treatment), the treatment is treatment of cancer. The cancer may be a cancer where the associated oncogene is known, or suspected to have, or be suitable for forming, a secondary or tertiary structure, or an oncogene expressing a nucleic acid having a secondary or tertiary structure, such as the quadruplex or pseudoknot structures described herein.

The disease for treatment, such as cancer, may be associated with the expression of, or altered regulation of (such as upregulation), neuroblastoma RAS (NRAS), metastasis associated lung adenocarcinoma transcript 1 (MALAT 1), EWS RNA Binding Protein 1 (EWSR1), telomeric repeat containing RNA (TERRA), B-cell lymphoma-extra large (BCL-XL), fibroblast growth factor receptor (FGFR), and MicroRNA 21 (MIR21), amongst others.

Patients Treated

In one embodiment (e.g., of use in methods of therapy, of use in the manufacture of medicaments, of methods of treatment), the treatment is administered to a subject in need of treatment.

The subject in need of treatment (the patient) may be a chordate, a vertebrate, a mammal, a placental mammal, a marsupial (e.g., kangaroo, wombat), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

The subject in need of treatment may be an adult or juvenile.

Preferably, the subject in need of treatment is a human, more preferably an adult human.

Alternatively, the subject in need of treatment is a non-human animal used in laboratory research. Preferably, the non-human animal is a rodent (e.g., a guinea pig, a hamster, a rat, a mouse).

### Routes of Administration

In one embodiment (e.g., of use in methods of therapy, of use in the manufacture of medicaments, of methods of treatment), the treatment is administered by any convenient route of administration, whether systemically/peripherally or topically (i.e., at the site of desired action).

The routes of administration may be oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Formulations

In one embodiment (e.g., of use in methods of therapy, of use in the manufacture of medicaments, of methods of treatment), the degrader of formula (I) is administered alone. Typically, however, it is preferable to present the degrader in a pharmaceutical formulation (e.g., composition, preparation, medicament) comprising at least one degrader as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents. The formulation may further comprise other active agents, for example, other therapeutic or prophylactic agents.

Thus, the present invention further provides pharmaceutical compositions, and methods of making a pharmaceutical composition comprising mixing at least one degrader described herein, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the compound.

The term “pharmaceutically acceptable” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Suitable carriers, diluents, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 5th edition, 25 2005.

The formulations may be prepared by any methods well known in the art of pharmacy. Such methods include the step of bringing into association the degrader with a carrier which constitutes one or more accessory ingredients. In general, the formulations are prepared by uniformly mixing the degrader with a carrier (e.g., a liquid carrier, a finely divided solid carrier, etc.), and then shaping the product, if necessary.

The formulation may be prepared to provide for rapid or slow release; immediate, delayed, timed, or sustained release; or a combination thereof.

Formulations may suitably be in the form of liquids, solutions (e.g., aqueous, nonaqueous), suspensions (e.g., aqueous, non-aqueous), emulsions (e.g., oil-in-water, water-in-oil), elixirs, syrups, electuaries, mouthwashes, drops, tablets (including, e.g., coated tablets), granules, powders, losenges, pastilles, capsules (including, e.g., hard and soft gelatin capsules), cachets, pills, ampoules, boluses, suppositories, pessaries, tinctures, gels, pastes, ointments, creams, lotions, oils, foams, sprays, mists, or aerosols.

Formulations may suitably be provided as a patch, adhesive plaster, bandage, dressing, or the like which is impregnated with one or more compounds and optionally one or more other pharmaceutically acceptable ingredients, including, for example, penetration, permeation, and absorption enhancers. Formulations may also suitably be provided in the form of a depot or reservoir.

The degrader may be dissolved in, suspended in, or mixed with one or more other pharmaceutically acceptable ingredients. The compound may be presented in a liposome or other microparticulate which is designed to target the compound, for example, to blood components or one or more organs.

### Dosages

In one embodiment (e.g., of use in methods of therapy, of use in the manufacture of medicaments, of methods of treatment), the treatment comprises administering a therapeutically-effective amount of a degrader of formula (I) to a subject in need of treatment.

It will be appreciated by one of skill in the art that appropriate dosages of the degraders described herein, and compositions comprising the degraders, can vary from patient to patient. Determining the optimal dosage will generally involve the balancing of the level of therapeutic benefit against any risk or deleterious side effects. The selected dosage level will depend on a variety of factors including, but not limited to, the activity of the particular degrader, the route of administration, the time of administration, the rate of excretion of the degrader, the duration of the treatment, other drugs, compounds, and/or materials used in combination, the severity of the disorder, and the species, sex, age, weight, condition, general health, and prior medical history of the patient. The amount of degrader and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the degrader is in the range of about 10 μg to about 250 mg (more typically about 100 μg to about 25 mg) per kilogram body weight of the subject per day.

Where the compound is a salt, an ester, an amide, a prodrug, or the like, the amount administered is calculated on the basis of the parent compound and so the actual weight to be used is increased proportionately.

### Other Preferences

Each and every compatible combination of the embodiments described above is explicitly disclosed herein, as if each and every combination was individually and explicitly recited.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

### EXAMPLES

Certain aspects and embodiments of the invention will now be illustrated by way of example and with reference to the figures described above.

### General Experimental Protocols

### In Vitro Pseudoknot Oligo Degradation Reactions

RNA oligo (20 μM) was added to a pH 7.5 HEPES (20 mM) buffer supplemented with KCl (50 mM) and EDTA (10 mM). The mixture was incubated at 37° C. for 30 min. MTDB-deg 16a, MTDB or TDB-deg 16b (1 mM) was then added. The reaction mixture was incubated at 37° C. for 3 h and then kept at 4° C. The reaction mixtures were analyzed by LC-MS or gel electrophoresis.

### LC-MS Analysis of Oligonucleotides.

Oligonucleotides were analyzed by LC-MS following the method of Mikutis et al., 2020.

Oligomers were analysed using a Xevo G2-S TOF mass spectrometer coupled to an Acquity UPLC system using an Acquity UPLC BEH C18 1.7 μm column. The system utilises electronspray (ESI) ionisation. Two mobile phases were used −16.3 mM TEA, 400 mM HFIP in H2O and 16.3 mM TEA, 400 mM HFIP in 80:20 v/v MeCN and H2O, with a flow rate of 0.200 mL/min. Calibration curves for the RNA species were based either on A260 or intensities of specified negative m/z signals. Intensities of integrated peaks were calculated using native modules of KNIME software platform (33). Total mass spectra were reconstructed from the ion series using the MaxEnt algorithm preinstalled on MassLynx software (v. 4.1 from Waters) according to the manufacturer's instructions. To obtain the negative ion series described, the oligomer peak in the chromatogram was selected for integration and further analysis.

### RNA Degradation Gel Electrophoresis

Gel electrophoresis was carried out following the method of Mikutis et al., 2020.

In vitro RNA degradation reactions were carried out as described above. The quenched reaction mixture was mixed in 1:1 ratio with a loading buffer (95% formamide, 0.025% SDS, 0.025% bromophenol blue (BPB), 0.025% xylene cyanol FF, 0.025% ethidium bromide, 0.5 mM EDTA), heated at 70° C. for 5 min, and cooled to 0° C. PAGE was performed on Novex™ TBEUrea Gels, containing 15% polyacrylamide under 1× TBE buffer (89 mM Tris, 89 mM boric acid, 2 mM EDTA) at 180 V for 60 min. Gel staining was performed using SYBR Green II RNA Gel Stain (Invitrogen) in 1× TBE buffer. The stained RNA was visualized with ChemiDoc MP (Bio-Rad, United Kingdom).

### Viral Stocks

SARS-CoV-2 stocks used to infect Vero CCL-81 cells were established from passage 4 of SARS-CoV-2 isolated from a Portuguese patient (internal reference: 606_IMM ID_5452) at approximately 1.7×10⁶ PFU/mL, after 4 days in Vero CCL-81 culture. Stock titers were calculated by plaque assay. Briefly, approximately 8×10¹ CCL-81 cells/well were seeded in 6-well plates and allowed to grow to confluence for 24 h. Medium was removed, and 500 μL of 10-fold serial dilutions of virus-containing supernatants were adsorbed in duplicate for 1 h, at 37 C. Plates were rocked manually to redistribute inoculum every 15 minutes. Cells were overlaid with 1.25% carboxymethylcellulose (CMC) in supplemented DMEM and incubated at 37° C. for 4 days. After incubation, the CMC overlay was removed, and cells were fixed with 4% formaldehyde/PBS and stained with 0.1% toluidine blue. After inactivation by fixation, plates were sealed with parafilm and disinfected before being removed from the BSC and BSL3. Viral plaques were counted to determine infectious titers (PFU (plaque forming units)/mL).

### Viral Infection of Vero CCL-81

Vero CCL-81 cells at 80% confluency were incubated with SARS-CoV-2 inoculum for 1 h at 37° C. After incubation, the inoculum was removed and DMEM medium supplemented with 2.5% FCS was added for 24 h, or until samples were harvested.

### Gel Electrophoresis Analysis

500 ng of SARS-CoV-2 RNA was incubated with or without 100 μM of MTDB-deg 16a in 1× HEPES buffer for 2 h at 37° C. with mild agitation. The samples were then analyzed on a 1.5% agarose gel.

### Nanopore Sequencing

500 ng of SARS-CoV-2 RNA was incubated with or without 100 μM of MTDB-deg 16a in 1× HEPES buffer for 2 h at 37° C. with mild agitation. The samples were then prepared for sequencing following the manufacturer's protocol for Direct RNA Sequencing (SQK-RNA002, ONT). The prepared libraries were loaded on FLO-MIN106D flow cells (ONT) and sequenced on a MinION Mk1C device (ONT).

Genomic sequence of the Wuhan-hu1 strain of SARS-CoV-2 (GenBank: MN908947.3) and the genomic annotation (NC_045512.2) were downloaded from the NCBI database. Sequence reads were aligned to the Wuhan-hu1 genome using minimap2 (Li et al., 2018). with parameters “-ax splice -N32-un -k13”. CIGAR strings of the alignments were processed by customized scripts. Reads were flagged as leader if the splice junction within the read starts between the first 60-120 bp of the genome. Reads were assigned to individual transcript if it covers more than 90% of the annotated transcript or more than 90% or the read sequence lies within the transcript.

### Drug Assay to Determine 50% Inhibitory Concentration

Increasing concentrations of MTDB-deg 16a (ranging from 0.07 to 25 μM) were tested to determine the 50% inhibitory concentration (IC₅₀). Vehicle (H₂O) control and control molecules were included in parallel. Cells were seeded in 96 well-plates at approximately 40% confluency 24 h before infection. MTDB-deg 16a, MTDB or TDB-deg 16b were added either 1 h before infection or 1 h after infection. SARS-CoV-2 cryopreserved stocks, were thawed at room temperature and used to infect cells at a 0.05 multiplicity of infection (MOI). Inhibition of viral growth was measured by harvesting cells at 24 h after infection. Viral growth was assessed by measuring viral loads by PCR targeting the E gene and pseudoknot region.

### Detection of Viral Plaque-Forming Units by Plaque Assay

Approximately 8×10⁵ CCL-81 cells/well were seeded in 6-well plates and allowed to grow to 80% confluence for 24 h. Supernatant of cultures treated with the compounds was diluted in DMEM medium supplemented with 2.5% FCS and added to pre-seeded wells of a 6-well plate and incubated for 1 h, at 37° C. Plates were rocked manually to redistribute inoculum every 15 min. Cells were overlaid with 1.25% CMC in supplemented DMEM and incubated at 37° C. for 4 days. After incubation, the CMC overlay was removed, and cells were fixed with 4% formaldehyde/PBS and stained with 0.1% toluidine blue. After inactivation by fixation, plates were sealed with parafilm and disinfected before being removed from the BSC and BSL3. Viral plaques were counted to determine infectious titers (PFU (plaque forming units)/mL).

### Quantification of Viral Load by PCR

Cell pellets were harvested into 300 μL of lysis buffer. Viral RNA was extracted by using a NZY Viral RNA Isolation kit (NZYtech) and cDNA was synthesized by using NZY First-Strand cDNA Synthesis kit (NZYtech) following manufactures' instructions. The quantitative RT-PCR (RT-qPCR) was then performed by using PowerUp SYBR Green Master Mix (BIO-RAD), set up by Applied Biosystems RT-PCR 7500 Fast machine with default SYBR green program.

The primers used for detecting SARS-CoV-2 were:

E gene:
5′-ACAGGTACGTTAATAGTTAATAGCGT-3′(forward),
5′-ATATTGCAGCAGTACGCACACA-3′(reverse);
N gene:
5′-GACCCCAAAATCAGCGAAAT-3′(forward),
5′-TCTGGTTACTGCCAGTTGAATCTG-3′(reverse);
Pseudo-knot:
5′-CCGCGAACCCATGCTTCAGTCA-3′(forward),
5′-CACGGTGTAAGACGGGCTGCAC-3′(reverse);
18S:
5′-GTAACCCGTTGAACCCCATT-3′(forward),
5′-CCATCCAATCGGTAGTAGCG-3′(reverse).

### Viral Recovery Assay

Two sets of samples were prepared for the recovery assay, where cells at 80% confluency were infected with SARS-CoV-2 cryopreserved stocks at a 0.05 MOI for 2 h. Then, the inoculum was removed, and infected cells were incubated with the MTDB-deg 16a, MTDB, and TDB-deg 16b at 6 μM for 24 h, at 37° C. and 5% CO₂. After 24 h, in one set of samples cells were harvested into lysis buffer for PCR analysis of viral growth. On the other set of samples, compounds in supernatant were removed, replaced by drug-free media and incubated for an additional 24 h, at 37° C. and 5% CO₂. After 24 h of incubation (corresponding to 48 h timepoint) cells were harvested into lysis buffer and viral growth was be measured by PCR targeting the E gene and pseudoknot region. Percentage of viral recovery was normalised to vehicle control.

### Cytotoxicity Assay

To determine if the compounds were toxic to cells, 1×10⁴ Vero E6 cells per well were seeded in 96-well plates. After 24 h, cells were incubated with increasing concentrations of MTDB-deg 16a, MTDB or TDB-deg 16b (ranging from 0.05 μM to 25 μM). The viability of cells after 24 h of incubation with the compounds was assessed by using CellTiter Blue viability assay (Promega) in accordance with manufacturer's protocol. Briefly, Cell titer blue stock solution was diluted 1:20. A volume of 80 μL of diluted cell titer blue was added to each well and incubated at 37° C., for 2 h.

### Dynamic Light Scattering (DLS)

Stock solutions (10 mM) of each screening molecule were prepared in neat DMSO and sequentially diluted in water to a final concentration of 25 or 12.5 PM. Data were collected on a Zetasizer Nano S (Malvern) at 25° C.

Antiviral activity on an animal model of SARS-CoV-2 infection

Ten to twelve week-old specific pathogen-free Hemizygous for Tg(K18-ACE2)2Prlmn (Strain B6.Cg-Tg(K18-ACE2)2Prlmn/J, the Jackson laboratory strain 034860) mice were used in this study. Mice were intranasally infected with 1×10⁴ PFU of SARS-CoV-2 in 50 μl of PBS. Compounds were administrated intranasally 1 hour pre-infection and 3 hours post-infection. Mice were treated either with vehicle (n=6), MTDB-degrader 16a at 25 mg/kg (n=6), MTDB at 10 mg/kg (n=3) or TDB-degrader 16b at 25 mg/kg (n=5). On day 5 post SARS-CoV-2 infection, animals were humanely euthanized and left lung was harvested for viral quantification by plaque assay and right lung was harvested for histopathological analysis.

### Western Blot Analysis.

For in vitro experiments, samples were treated with vehicle (H₂O) or MTDB-deg 16a (6 mM) for 24 hours. Cells were then lysed using whole cell lysis buffer (50 mM Tris-HCl pH=8.0, 450 mM NaCl, 0.1% NP-40, 1 mM EDTA), supplemented with 1 mM DTT, protease inhibitors (Sigma), and phosphatase inhibitors (Sigma). For in vivo experiments, the whole left lung from mice was homogenized in 3 mL of DMEM and 750 μL was transferred to an equal volume of whole cell lysis buffer, supplemented as above. Protein concentrations were accessed using Bradford Assays (BioRad). Prior to loading the samples were supplemented with LDS Loading Buffer (Life technologies) and Sample Reducing Agent (Life Technologies). 40 μg of protein was separated on SDS-PAGE gels and blotted onto polyvinylidene difluoride (PVDF) membranes (GE Healthcare). Western Blot experiments were performed using the following antibodies: anti-beta actin (Abcam, ab8224), anti phospho-MAPKAPK-2 (Thr334) (27B7) (Cell Signalling, 3007), anti-Phospho-p38 MAPK (Thr180/Tyr182) (D3F9) XP@ (Cell Signalling, 4511), goat anti-mouse IgG H&L (HRP) (Abcam, ab205719) and goat-anti rabbit HRP (Abcam, ab6721).

### Synthesis of Azido-Imidazoles

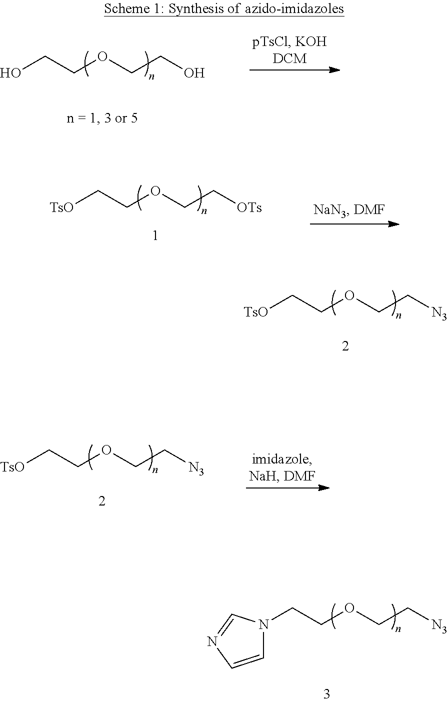

### Synthesis of Hexaethylene glycol di(p-toluenesulfonate) (1)

Hexaethylene glycol (1.0 mmol) was solved in DCM (10 mL) and p-toluenesulfonyl chloride (2.2 mmol) and KOH (10 mmol) were added at 0° C. The reaction mixture was stirred for 6 hours at room temperature, then filtered and washed with water. After drying over MgSO₄, the solvent was evaporated under reduced pressure. No further purification necessary. Yield: 95% (colourless oil).

¹H NMR (400 MHz, CDCl₃): δ_{H} 7.78 (d, 4H), 7.33 (d, 4H), 4.14 (t, 4H), 3.67 (br tr, 4H, 3.60 (br s, 8H), 3.57 (br s, 8H), 2.43 (br s, 6H). MS: m/z for C₂₆H3₉O₁₁S2: 591.19.

The physical and spectroscopic data were in agreement with that described in the literature (Mikutis et al., 2020).

### Synthesis of Hexaethylene glycol p-toluenesulfonate azide (2a)

Hexaethylene glycol di(p-toluenesulfonate) 1 (1.0 mmol) was dissolved in DMF (10 mL) and sodium azide (1.0 mmol) was added. The reaction mixture was stirred at 60° C. for 6 hours, then cooled down to room temperature and stirred over-night. The mixture was washed with brine and dried over MgSO₄. To remove DMF, toluene was added and the solvent was evaporated under reduced pressure. The crude product was purified via column chromatography (EtOAc: Hexane, 1:1). Yield: 54% (colourless oil).

¹H NMR (400 MHz, CDCl₃) δ_{H} 7.82 (d, 2H), 7.36 (d, 2H), 4.18 (t, 2H), 3.59-3.73 (20H, PEG), 3.41 (t, 2H), 2.47 (s, 3H). MS: m/z for C₁₉H3₁N3NaO₈S: 484.2.

The physical and spectroscopic data were in agreement with that described in the literature (Mikutis et al., 2020).

### Synthesis of Tetraethylene glycol p-toluenesulfonate azide (2b)

Tetraethylene glycol di(p-toluenesulfonate) (2.7 g, 5.4 mmol) was dissolved in anhydrous DMF (10 ml). Sodium azide (355 mg, 5.4 mmol) was added and the mixture was placed under N₂ and stirred for 18 h at 55° C. The solvent was removed in vacuo, and the products were purified via flash column chromatography (3:1 Pet. Ether:AcOEt to 1:1 Pet. Ether:AcOEt). The product was obtained as a colourless oil (798 mg, 2.1 mmol, 39%).

¹H NMR (400 MHz, CDCl3) δ 7.82 (d, 2H), 7.37 (d, 2H), 4.19 (t, 1H), 3.60-3.73 (12H, PEG), 3.40 (t, 2H), 2.47 (s, 3H). MS: m/z for C₁₅H₂₃N₃NaO₆S 396.1207.

### Synthesis of diethylene glycol p-toluenesulfonate azide (2c)

Diethylene glycol di(p-toluenesulfonate) (1.0 mmol) was dissolved in DMF (10 mL) and sodium azide (1.0 mmol) was added. The reaction mixture was stirred at 60° C. for 6 hours, then cooled down to room temperature and stirred over-night. The mixture was washed with brine and dried over MgSO₄. To remove DMF, toluene was added and the solvent was evaporated under reduced pressure. The crude product was purified via column chromatography (EtOAc: Hexane, 1:1). Yield: 59% (colourless oil).

¹H NMR (400 MHz, CDCl₃) δ_{H} 7.80 (d, 2H), 7.35 (d, 2H), 4.17 (t, 2), 3.70 (t, 2H), 3.61 (t, 2H), 3.32 (t, 2H), 2.45 (s, 3H). MS: m/z for C₁₁H₁₅N₃NaO₄S: 308.068.

The physical and spectroscopic data were in agreement with that described in the literature (Mikutis et al., 2020).

### Synthesis of hexaethylene glycol imidazolate azide (3a)

Imidazole (1.0 mmol) was dissolved under inert conditions in dry DMF (15 mL) and sodium hydride (60% dispersion in mineral oil, 1.2 mmol) was added. After stirring for 30 min at 0° C., 2a (1.0 mmol) was added. The reaction mixture was stirred at 60° C. overnight and, after cooling to room temperature, the mixture was quenched with water (20 mL). Subsequent extraction with EtOAc and DCM, drying over MgSO₄ and evaporation of the solvent under reduced pressure gave the crude product. The crude product was then purified via column chromatography (EtOAc: MeOH, 3:1). Yield: 35% (colourless oil).

¹H NMR (400 MHz, CDCl₃) δ_{H} 7.52 (s, 1H), 7.02 (s, 1H), 6.98 (s, 1H), 4.09 (t, 2H), 3.72 (t, 2H), 3.55-3.78 (18H, PEG), 3.36 (t, 2H). MS: m/z for C₁₅H₂₈N₅O₅ 358.21.

The physical and spectroscopic data were in agreement with that described in the literature (Mikutis et al., 2020).

### Synthesis of Tetraethylene Glycol Imidazolate Azide (3b)

Imidazole (18 mg, 0.27 mmol) and NaH (60% dispersion in mineral oil, 12 mg, 0.27 mmol) were suspended in anhydrous DMF (1 ml) at 0 C. The mixture was placed under N₂ atmosphere, allowed to warm to room temperature and stirred for 30 min. 2b (100 mg, 0.27 mmol) was dissolved in anhydrous DMF (1 ml) and the resulting solution was added to the first mixture. It was then stirred for 20 h at 55° C. Solvent was then removed in vacuo and the resulting residue was purified via flash chromatography (dry loading, gradient EtOAC to 9:1 EtOAc: MeOH). The product was obtained as a colourless oil (54 mg, 0.20 mmol, 74%).

¹H NMR (400 MHz, CDCl₃) δ 7.55 (s, 1H), 7.05 (s, 1H), 7.05 (s, 1H), 4.12 (t, 2H), 3.75 (t, 2H), 3.60-3.71 (10H, PEG), 3.39 (t, 2H). ¹¹C NMR (100 MHz, CDCl₃) δ_{c} 137.6, 129.2, 119.4, 70.5-70.7 (multiple PEG peaks), 70.0, 50.7, 47.1. MS: m/z for C˜ H₁₉N5O₃ 270.1582.

Synthesis of Diethylene glycol imidazolate azide (3c) Imidazole (1.0 mmol) was dissolved under inert conditions in dry DMF (15 mL) and sodium hydride (60% dispersion in mineral oil, 1.2 mmol) was added. After stirring for 30 min at 0° C., 2c (1.0 mmol) was added. The reaction mixture was stirred at 60° C. overnight and then cooled to room temperature. The mixture was quenched with water (20 mL). After extraction with EtOAc and DCM, drying over MgSO₄, and evaporation of the solvent under reduced pressure, a crude product was obtained. The crude product was then purified via column chromatography (EtOAc: MeOH, 3:1). Yield: 38% (colourless oil).

¹H NMR (400 MHz, CDCl₃) δ_{H} 7.53 (s, 1H), 7.06 (s, 1H), 6.99 (s, 1H), 4.14 (t, 2H), 3.75 (t, 2H), 3.60 (t, 2H), 3.36 (t, 2H). MS: m/z for C˜ H₁₉N5O₃ 182.1

The physical and spectroscopic data were in agreement with that described in the literature (Mikutis et al., 2020).

### Synthesis azido-ethyl imidazole (4)

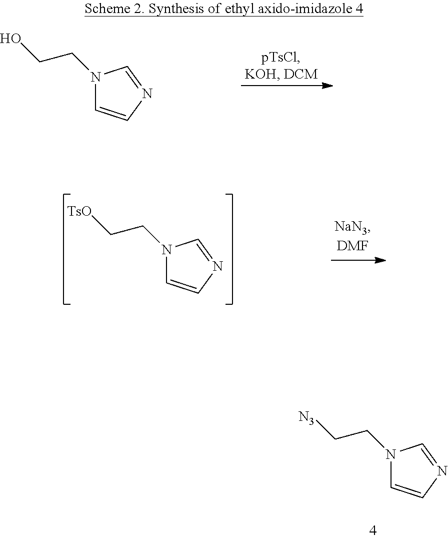

Hydroxyethyl imidazole (1.0 mmol) was dissolved in DCM (10 mL) at 0° C. and KOH (10 mmol) and p-toluenesulfonyl chloride (1.2 mmol) were added. The reaction mixture was stirred for 6 hours at room temperature, then filtered and the solvent was removed under reduced pressure. The crude was resolved in DMF and sodium azide (1.0 mmol) was added.

It was stirred at 60° C. for 6 hours, then cooled down to room temperature and stirred over-night. Toluene was added and the solvent was removed under reduced pressure. Purification was performed via column chromatography (EtOAC: MeOH, 3:1). Yield: 26% (white solid).

¹H NMR (400 MHz, CDCl₃) δ 7.51 (s, 1H), 7.09 (s, 1H), 6.96 (s, 1H), 4.09 (t, J=5.7 Hz, 1H), 3.62 (t, J=5.7 Hz, 1H).

### Synthesis of Pyridostatin Degraders

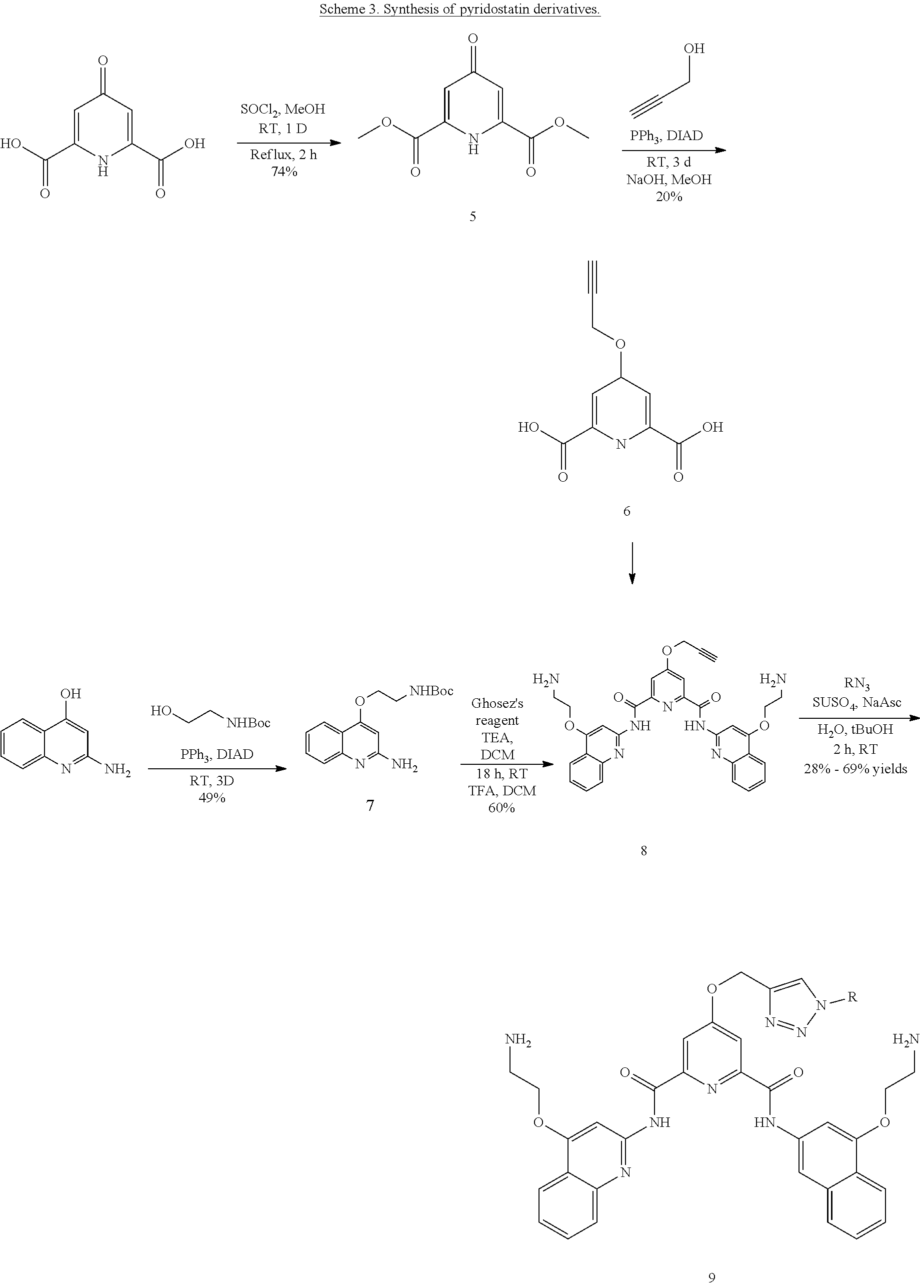

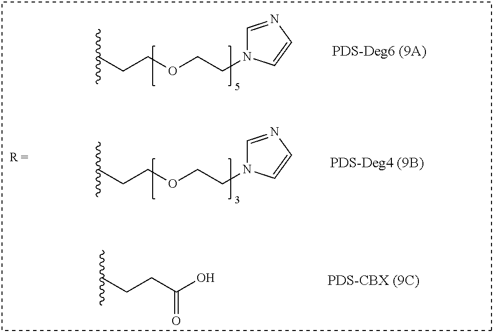

### Synthesis of Chelidamic acid dimethyl ester (5)

Chelidamic acid hydrate (2.0 g, 11 mmol) was suspended in 20 ml MeOH. Thionyl chloride (500 μL, 6.9 mmol) was added dropwise to the suspension at −10° C. A white to brown colour change was observed. The solution was warmed to RT and stirred overnight. The brown solution was refluxed for 2 h and the solvent removed in vacuo. The brown crude product was then re-crystallised from EtOH, resulting in a beige solid chelidamic acid dimethyl ester 5 (864 mg, 3.9 mmol, 36%).

¹H NMR (400 MHz, DMSO) δ 11.77 (br s, 1H), 7.61 (s, 2H), 3.88 (s, 6H). ¹³C NMR (101 MHz, DMSO) δ 165.97, 164.88, 149.37, 115.33, 52.68. HRMS (ES) calculated for C₉H₁₀NO₅ ([M+H]+) m/z: 212.0559, found 212.0567.

### Synthesis of Propargylic Chelidamic Acid (6)

Chelidamic acid dimethyl ester 5 (0.82 g, 3.8 mmol), propargyl alcohol (0.33 mL, 5.7 mmol) and polymer bound triphenylphosphine (3.47 g, 1.5 mmol loading/g, 5.2 mmol) were suspended in 55 mL freshly distilled THF. The solution was degassed using freeze-pump-thaw cycling and cooled to 0° C., DIAD (1.0 mL, 5.1 mmol) was added dropwise under argon.

The solution was warmed to RT and stirred for 3d. The solution was filtered, and the solvent removed in vacuo. Chelidamic acid dimethyl ester was obtained via column chromatography (50% EtOAc, 50% pet. ether). It was then was dissolved in 50 mL MeOH, followed by addition of 50 mL aqueous NaOH (0.33 g, 7.5 mmol) solution. The resulting mixture was stirred for 5 min and deprotection was confirmed by TLC. The organic solvent was removed in vacuo. 5% formic acid was added to acidify followed by extraction with EtOAC 3×100 mL. The organic layer was then dried with MgSO₄, filtered and the solvent removed in vacuo. This yielded an off-white solid propargylic chelidamic acid 6 (0.17 g, 0.77 mmol, 20%).

¹H NMR (400 MHz, MeOD) δ 7.93 (s, 2H), 5.02 (d, J=2.4 Hz, 2H), 3.15 (t, J=2.5 Hz, 1H). ¹³C NMR (100 MHz, MeOD) δ 168.12, 166.98, 150.54, 150.43, 115.73, 78.97, 77.90, 77.88, 57.74, 57.68. HRMS (ES) calculated for C₁₀H8NO₅ ([M+H]+) m/z: 222.0397, found 222.0391.

### Synthesis of O-(Ethyl-2-N-boc-amine)-2-aminoquinolinone (7)

2-aminoquinolinone (1.0 g, 6.2 mmol), N-boc ethanolamine (1.4 mL, 9.1 mmol) and triphenylphosphine (3.3 g, 13 mmol) were dissolved in 10 mL freshly distilled THF. The solution was degassed using freeze-pump-thaw cycling and cooled to 0° C., DIAD (1.8 mL, 9.2 mmol) was added dropwise under argon. The solution warmed to RT and stirred for 3d. The solvent was then removed in vacuo. The product was purified by a gradient column chromatography with 100% EtOAc to 90% EtOAc, 10% MeOH. The solvent was removed in vacuo to obtain an off-white solid 7 (552 mg, 1.82 mmol, 29%).

¹H NMR (400 MHz, CDCl₃) δ 7.98 (dd, J=8.0, 1.0 Hz, 2H), 7.60 (dd, J=8.4, 1.2 Hz, 2H), 7.55 (ddd, J=8.5, 6.7, 1.6 Hz, 2H), 7.23 (ddd, J=8.1, 6.6, 1.3 Hz, 2H), 6.04 (s, 1H), 5.01 (br s, 1H), 4.69 (br s, 2H), 4.18 (t, J=5.1 Hz, 4H), 3.68 (q, J=5.5 Hz, 4H), 1.46 (s, 9H). ¹³C NMR (100 MHz, CDCl₃) δ 162.32, 158.13, 155.90, 148.55, 130.25, 125.63, 121.97, 121.60, 117.52, 90.09, 79.83, 67.52, 39.82, 28.38. HRMS (ES) calculated for C₁₆H2₂N30₃ ([M+H]+) m/z: 304.1661, found 304.1649.

### Synthesis of Alkyne-Pyridostatin (8)

Propargylic chelidamic acid 6 (0.12 g, 0.54 mol) was dissolved in 1.2 mL DCM. Then Ghosez reagent (170 μL, 1.3 mmol) was added dropwise at 0° C. The orange solution was then stirred at RT for 2 h. The chlorination was confirmed by TLC. Triethylamine (0.18 mL, 1.3 mmol) was added dropwise at 0° C. The solution was then stirred at RT for 1 h. 7 (0.37 g, 1.2 mmol) was suspended in 1.2 mL DCM and then added dropwise to the mixture. The mixture turned brown-red and was stirred under argon overnight. The crude protected product 8a (not shown) was precipitated from hot MeCN as a red solid. The red solid 8a was then dissolved in DCM. A 2:1 mixture of DCM:TFA was added to acidify the solution and remove the N-boc protection. The solvent was removed in vacuo and the product was purified via HPLC (gradient 100% H₂O, 0.1% FA to 100% MeCN, 0.1% FA). Lyophilization afforded an off-white solid Alkyne-Pyridostatin 8 (51 mg, 86 μmol, 16%).

HRMS (ES) calculated for C₃₂H3₀N70₅ ([M+H]+) m/z: 592.2308, found 592.2327.

### Synthesis of Pyridostatin Degraders (9A to 9C)

Alkyne-Pyridostatin 8 (15 mg, 25 μmol) was dissolved in 2.5 mL of a 2:1 mixture of H₂O: tBuOH. A solution of copper sulfate pentahydrate (250 μL, 100 mM, 25 μmol) was added followed by a solution of sodium ascorbate (1.3 mL, 100 mM, 130 μmol). The cloudy yellow solution was degassed and stirred for 10 mins. A solution of the appropriate azido-imidazole (3a, 3b or 3-azidopropionic acid) (3.8 mL,10 mM) was then added. The solution was stirred under argon for 2 h. The organic solvent was removed in vacuo. Then the product was purified via HPLC (gradient 100% H₂O, 0.1% FA to 100% MeCN, 0.1% FA). The product was obtained as a white or an off-white solid.

9A (PDS-deg6). 48% yield (11.3 mg, 12 μmol). HRMS (ES) calculated for C₄₇H52N12010 ([M+H]+) m/z: 949.4321, found 949.4344.

9B (PDS-deg4). 69% yield (14.8 mg, 17 μmol). HRMS (ES) calculated for C₄₃H49N1208 ([M+H]+) m/z: 861.3796, found 861.376.

9C (PDS-CBX). 28% yield (4.9 mg, 6.9 μmol). HRMS (ES) calculated for C₃₅H34N1007 ([M+H]+) m/z: 707.2690, found: 707.2684.

### Synthesis of Pseudoknot-Degrader

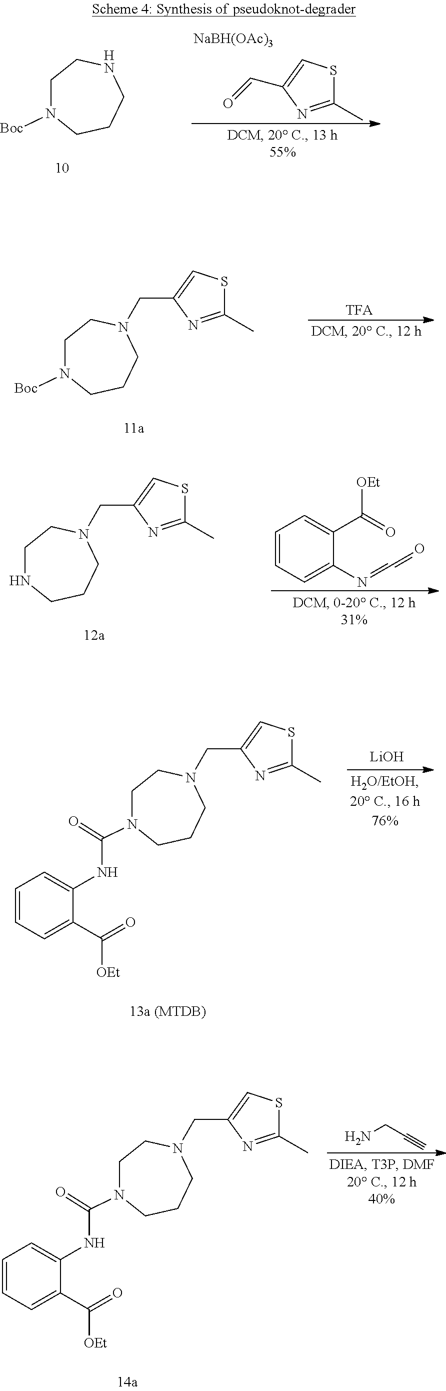

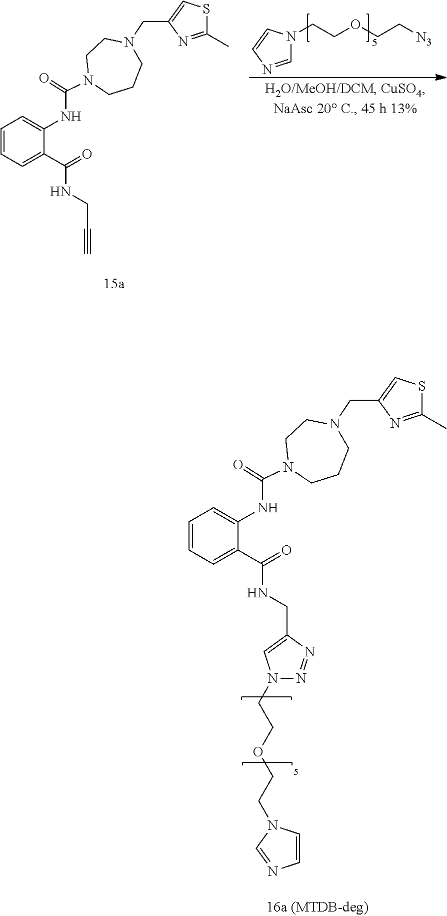

Synthesis of compound 11a

2-methylthiazole-4-carbaldehyde (10.0 g, 78.6 mmol, 1.0 equiv.) in DCM (100 mL) was added to compound 10 (16.5 g, 82.6 mmol, 16.2 mL, 1.1 equiv.) in one portion at 25° C. under N₂.

The mixture was stirred at 25° C. for 3 h. To the mixture was added NaBH(OAc)₃ (25.0 g, 118 mmol, 1.5 equiv.) and stirred for 10 h. The residue was poured into water (50 mL) and stirred for 10 min. The aqueous phase was extracted with DCM (3×20 mL). The combined organic phase was dried with anhydrous Na₂SO₄ and filtered; the solvent was removed in vacuo. The product was purified via column chromatography (gradient, petroleum ether to petroleum ether/ethyl acetate 10/1) to yield compound 11a (13.5 g, 43.4 mmol, 55% yield) as a yellow oil.

LCMS [+scan]: calculated m/z C₂₀H2₆N30₃S 388.2; observed 388.1.

Synthesis of compound 12a

TFA (40.0 g, 351 mmol, 26 mL, 8.4 equiv.) was added to compound 11a (13.0 g, 41.7 mmol, 1.0 equiv.) in DCM (130 mL) at 25° C. at N₂. The mixture was stirred for 12 h. The solvent was removed in vacuo to give a TFA salt of compound 12a (23.0 g, crude) as a red oil.

LCMS [+scan]: calculated m/z C₁₀H11N₃S 212.1; observed 212.0.

Synthesis of MTDB (compound 13a)

To a solution of compound 12a (20.0 g, 45.5 mmol, 1.0 equiv.) in DCM (200 mL) was added TEA (9.21 g, 91.0 mmol, 12.7 mL, 2.0 equiv.) at 20° C. under N₂. Then ethyl 2-isocyanatobenzoate (8.70 g, 45.5 mmol, 1.0 equiv.) was added to the mixture at 0° C. The mixture was stirred at 20° C. for 12 h. The solvent was removed in vacuo. The residue was purified by column chromatography (gradient, petroleum ether/ethyl acetate 100/1 to 20/1) to give MTDB (compound 13a 5.62 g, 14.0 mmol, 31% yield) as an off-white solid.

¹H NMR (400 MHz, CD30D): δ 8.42 (br d, J=8.4 Hz, 1H), 8.08 (br d, J=8.0 Hz, 1H), 7.62 (s, 1H), 7.52-7.59 (m, 1H), 7.09 (br t, J=7.6 Hz, 1H), 4.34-4.46 (m, 4H), 3.93 (br s, 2H), 3.77 (br t, J=6.0 Hz, 2H), 3.49 (br s, 4H), 3.28-3.31 (m, 1H), 2.74 (s, 3H), 2.31 (br d, J=4.8 Hz, 2H), 1.43 (t, J=7.2 Hz, 3H). LCMS [+scan]: calculated m/z C₂₀H2₇N40₃S 403.2; observed 403.1.

Synthesis of compound 14a

To a mixture of MTDB (compound 13a 5.60 g, 13.9 mmol, 1.0 equiv.) in EtOH (120 mL) and H₂O (30 mL) was added LiOH monohydrate (2.34 g, 55.7 mmol, 4.0 equiv.) at 25° C. under N₂. The mixture was stirred at 25° C. for 12 h. The mixture was adjusted to pH 6 with 1 M HCl and the aqueous phase was extracted with ethyl acetate (3×40 mL); then the organic phase was dried with anhydrous Na₂SO₄, filtered and concentrate in vacuo to give compound 14a (2.60 g, 6.94 mmol, 50%) as a yellow solid.

LCMS [+scan]: calculated m/z C₁₈H2₃N40₃S 375.1; observed 375.1.

Synthesis of compound 15a

To a mixture of compound 14a (2.60 g, 6.94 mmol, 1.0 equiv.) and propargyl amine (1.15 g, 20.8 mmol, 1.33 mL, 3.0 equiv.) in DMF (200 mL) was added DIEA (4.49 g, 34.7 mmol, 6.05 mL, 5.00 equiv.) at 25° C. under N₂. The mixture was added T₃P (4.42 g, 13.9 mmol, 4.13 mL, 2.0 equiv.) and stirred at 50° C. for 12 h. The mixture was poured into water (200 mL) and the aqueous phase was extracted with ethyl acetate (3×70 mL); then the organic phase was washed with brine (60 mL); the organic phase was dried with anhydrous Na₂SO₄, filtered and the solvent was removed in vacuo. The residue was purified by column chromatography (gradient, petroleum ether/ethyl acetate 100/1 to ethyl acetate) to yield 15a (1.20 g, 2.80 mmol, 40% yield).

¹H NMR (400 MHz, CDCl₃): δ 10.54 (br s, 1H), 8.46 (d, J=8.2 Hz, 1H), 7.43-7.51 (m, 2H), 7.11-7.11 (m, 1H), 6.99 (q, J=7.6 Hz, 2H), 6.49 (br s, 1H), 4.22 (dd, J=5.2, 2.6 Hz, 2H), 3.72-3.83 (m, 4H), 3.64-3.69 (m, 2H), 2.77-2.96 (m, 4H), 2.72 (s, 3H), 2.32 (t, J=2.6 Hz, 1H), 2.04 (br d, J=15.2 Hz, 2H). LCMS [+scan]: calculated m/z C₂₁H₂₆N5O₂S 412.2; observed 412.0.

Synthesis of MTDB-deg (compound 16a)

A mixture of azido-imidazole 3a (200 mg, 280 μmol, 1.0 equiv.), compound 15a (115 mg, 280 μmol, 1.0 equiv.) and CuSO₄ (22.3 mg, 140 μmol, 21.5 μL, 0.5 equiv.) in DCM (5 mL), MeOH (5 mL) and H₂O (5 mL) were stirred at 20° C. for 0.5 h; then NaAsc (11.1 mg, 55.9 μmol, 0.2 equiv.) was added to the mixture and stirred at 20° C. for 4.5 h. The mixture was diluted with H₂O (10 mL) and then extracted with DCM (3×10 mL). The combined organic phase was dried over anhydrous Na₂SO₄, filtered and concentrated in vacuo. The residue was purified by via HPLC (column: Phenomenex Gemini-NX 80×40 mm×3 um; mobile phase: [water (10 mM NH₄HCO₃)-ACN]; B %: 15%-35%, 8 min) to give MTDB-deg 16a (28.0 mg, 34.9 μmol, 13% yield) as a light yellow oil.

¹H NMR (400 MHz, DMSO-d₆): δ 11.03 (s, 1H), 9.29 (br t, J=5.2 Hz, 1H), 8.37 (d, J=8.4 Hz, 1H), 7.96 (s, 1H), 7.74 (br d, J=7.2 Hz, 1H), 7.63 (br s, 1H), 7.43 (t, J=7.60 Hz, 1H), 7.15-7.31 (m, 2H), 6.99 (t, J=7.6 Hz, 1H), 6.89 (br s, 1H), 4.46-4.53 (m, 4H), 4.10 (br t, J=5.2 Hz, 2H), 3.77-3.81 (m, 2H), 3.71 (br s, 2H), 3.66 (br t, J=5.07 Hz, 2H), 3.44-3.57 (m, 20H), 3.34 (s, 25H), 2.73 (br s, 1H), 2.62 (br s, 6H), 1.84 (br s, 2H). HRMS [+scan]: calculated m/z C₃₆H₅₃N₁₀O₇S 769.3819; observed 769.3830.

### Synthesis of Control Degrader for Pseudoknot Experiments

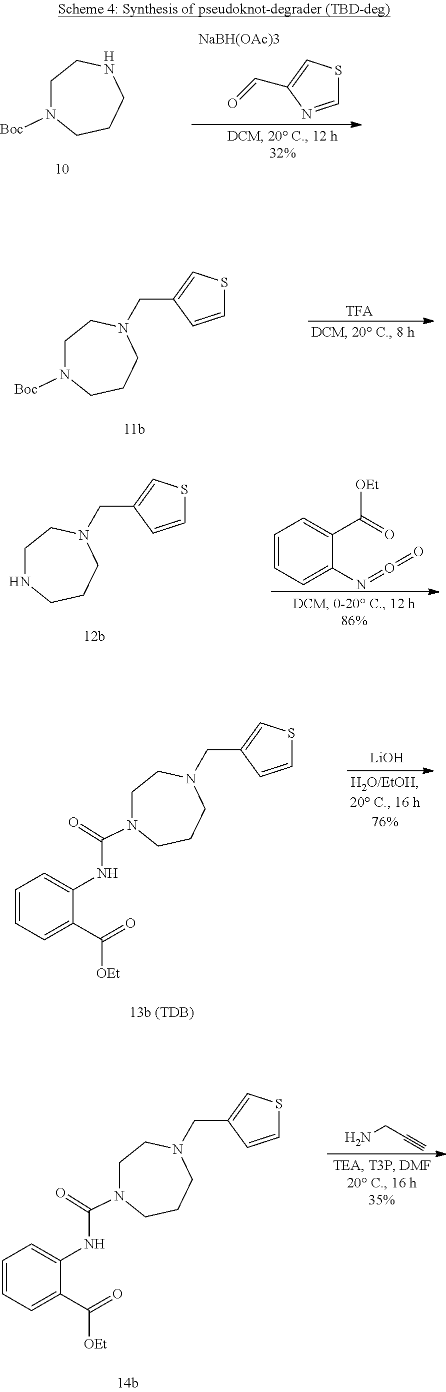

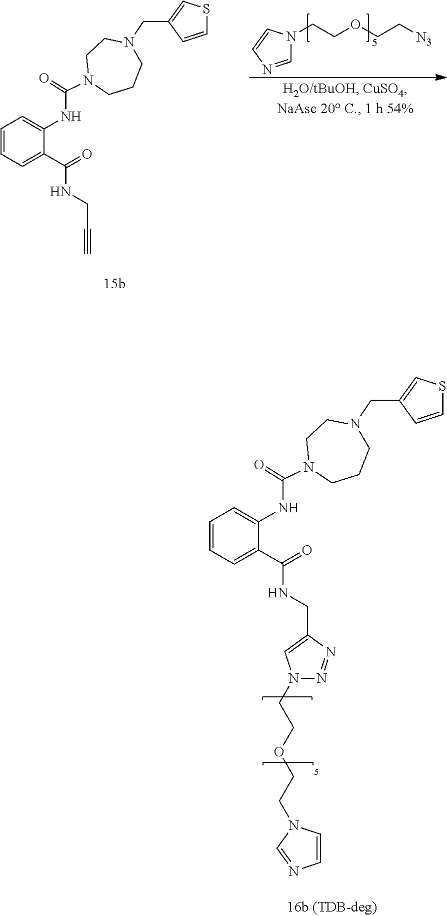

Synthesis of compound 11b

To thiophene-3-carbaldehyde (2.00 g, 17.8 mmol, 1.63 mL, 1.0 equiv.) in DCM (80 mL) was added compound 10 (3.93 g, 19.6 mmol, 3.85 mL, 1.1 equiv.) at 20° C. under N₂. The mixture was stirred at 20° C. for 3 h. Then NaBH(OAc)₃ (5.67 g, 26.8 mmol, 1.5 equiv.) was added to the mixture at 0° C. and stirred at 20° C. for 10 h. The reaction mixture was quenched by addition of water (60 mL) and extracted with DCM (2×20 mL). The combined organic layers were washed with brine (30 mL), dried over anhydrous Na₂SO₄, filtered and concentrated in vacuo. The residue was purified by silica gel chromatography (gradient petroleum ether/ethyl acetate=100/1 to 20/1) to give compound 11b (1.70 g, 5.73 mmol, 32% yield) as a red oil.

¹H NMR (400 MHz, CDCl₃): δ 7.21-7.31 (m, 1H), 7.03-7.15 (m, 2H), 3.64 (s, 2H), 3.37-3.54 (m, 4H), 2.55-2.68 (m, 4H), 1.81 (br dd, J=10.8, 4.9 Hz, 2H), 1.40-1.52 (m, 9H).

Synthesis of compound 12b

To a solution of compound 11 b (1.70 g, 5.73 mmol, 1.0 equiv.) in DCM (20 mL) was added TFA (6.16 g, 54.0 mmol, 4.00 mL, 9.4 equiv.) at 20° C. under N₂. The mixture was stirred at 20° C. for 8 h. The solvent was removed in vacuo to yield a TFA salt of compound 12b (3.00 g, crude) as a red oil which was used in the next step without further purification.

Synthesis of TDB (compound 13b)

To a mixture of compound 12b (3.00 g, 9.67 mmol, 1.0 equiv.) in DCM (40 mL) was added TEA (1.96 g, 19.3 mmol, 2.69 mL, 2.0 equiv.) at 20° C. under N₂. Then ethyl 2-isocyanatobenzoate (1.85 g, 9.67 mmol, 1.0 equiv.) was added to the mixture at 0° C. The mixture was stirred at 20° C. for 12 h. The solvent was removed in vacuo. The residue was purified by column chromatography (gradient, Petroleum ether/Ethyl acetate=100/1 to 20/1) to give TDB 13b (3.30 g, 8.35 mmol, 86% yield) as an off-white solid.

¹H NMR (400 MHz, CDCl₃): δ 10.59 (s, 1H), 8.52 (d, J=8.4 Hz, 1H), 7.94 (dd, J=8.0, 1.53 Hz, 1H), 7.42 (t, J=7.6 Hz, 1H), 7.14-7.24 (m, 1H), 6.96-7.07 (m, 2H), 6.88 (t, J=7.2 Hz, 1H), 4.28 (q, J=7.2 Hz, 2H), 3.55-3.67 (m, 6H), 2.70 (br s, 2H), 2.53-2.65 (m, 2H), 1.91 (br s, 2H), 1.33 (t, J=7.2 Hz, 3H). LCMS [+scan]: calculated m/z C₂₀H₂₆N₃O₃S 388.2; observed 388.1.

Synthesis of compound 14b

To a mixture of TDB 21 (100 mg, 0.26 mmol, 1.0 equiv.) in EtOH (1.2 mL) and H₂O (1.2 mL) was added LiOH monohydrate (65 g, 1.5 mmol, 6.0 equiv.) at 25° C. under N₂. The mixture was stirred at 25° C. for 16 h, after which time additional LiOH monohydrate (130 g, 3.1 mmol, 12.0 equiv.) was added. After 2 h the mixture was adjusted to pH 6 with 1 M HCl and the aqueous phase was extracted with DCM (3×10 mL); then the organic phase was dried with anhydrous MgSO₄, filtered and concentrate in vacuo to give compound 14b (70 mg, 0.19 mmol, 76%) as a yellow oily solid.

¹H NMR (400 MHz, CD30D): b_{H} 8.41 (d, J=8.5 Hz, 1H), 8.08 (d, J=8.5 Hz, 1H), 7.76 (br s, 1H), 7.64 (m, 1H), 7.53 (t, J=7.5 Hz, 1H), 7.32 (d, J=4.5 Hz, 1H), 7.07 (t, J=7.5 Hz, 1H), 4.46 (br s, 2H), 3.95 (m, 2H), 3.75 (tr, J=6.0 Hz, 2H), 3.47 (m, 4H), 2.34 (m, 2H). ¹³C NMR (100 MHz, CD30D) δ_{H} 170.9, 155.4, 142.7, 133.7, 131.2, 129.7, 129.3, 128.8, 127.6, 121.2, 119.0, 115.3, 55.2, 54.9, 53.3, 44.5, 40.2, 24.0. HRMS [+scan]: calculated m/z C₁₋₃H₂₂N₃O₃S 360.1382; observed 360.1386.

Synthesis of compound 15b

To a mixture of compound 14b (70 mg, 195 μmol, 1.0 equiv.) and propargyl amine (11.2 mg, 200 μmol, 13.1 μL, 1.0 equiv.) in DMF (3 mL) was added TEA (75.5 mg, 74.6 μmol, 104 μL, 4.0 equiv.) at 25° C. under N₂. The mixture was added 50% T₃P in DMF (238 mg, 370 μmol, 1.9 equiv.) and stirred at 25° C. for 16 h. The solvent was removed in vacuo and the title compound was purified on a column (gradient DCM to DCM:MeOH 9:1). To remove residual DMF, the compound was dissolved in DCM (10 mL) and washed with H₂O (10 mL) and 1% aqueous NaOH solution (10 mL). The organic phase was dried with anhydrous MgSO₄, the solvent was removed in vacuo to afford 15b (26 mg, 66 μmol, 34% yield, ratio of 4:1).

¹H NMR (400 MHz, CDCl₃, reported for major diastereomer): δ 10.42 (br s, 1H), 8.35 (d, J=8.9 Hz, 1H), 7.36-7.41 (m, 2H), 7.26 (m, 1H), 7.11 (m, 1H), 7.06 (m, 1H), 6.87-6.97 (m, 2H), 6.49 (br s, 1H), 4.16 (dd, J=5.2, 2.5 Hz, 2H), 3.59-3.71 (m, 6H), 2.75 (br s, 2H), 2.65 (t, J=5.5 Hz, 2H), 2.27 (t, J=2.5 Hz, 1H), 1.96 (br s, 2H). ¹³C NMR (100 MHz, CDCl₃) bo 169.4, 155.4, 141.5, 140.0, 132.5, 128.4, 126.8, 125.5, 122.7, 121.1, 120.9, 118.9, 79.2, 71.8, 57.4, 55.0, 46.0, 29.6. HRMS [+scan]: calculated m/z C₃₆H5₂N90₇S 397.1698; observed 397.1716.

Synthesis of TDB-deg (compound 16b)

Compound 15b (9.9 mg, 25 μmol, 1.0 equiv.) was dissolved in a mixture of H₂O (1.7 mL) and tBuOH (0.8 mL). Aqueous CuSO₄ solution (250 μL, 100 mM, 25 μmol, 1.0 equiv.) was added, followed by aqueous NaAsc solution (1.3 mL, 100 mM, 130 μmol, 5.2 equiv.). The resulting cloudy yellow mixture was put under argon atmosphere; aqeous solution of azido-imidazole 3a (3.8 mL, 10 mM, 38 μmol, 1.5 equiv.) was then added. The reaction was stirred at room temperature for 1 h, after which they reation mixture turned clear yellow. The reaction was quenched with disodium EDTA dihydrate (9.3 mg, 25 μmol, 1 equiv.), the organic solvent was removed in vacuo and the mixture was purified via HPLC. The fractions containing the product were lyophilised, resulting in TDB-deg (15b) as a yellow-brown oily solid (10.2 mg, 14 μmol, 54% yield).

HRMS [+scan]: calculated m/z C₃₆H5₂N90₇S 754.3710; observed 754.3698.

### Synthesis of Chloramphenicol Degraders

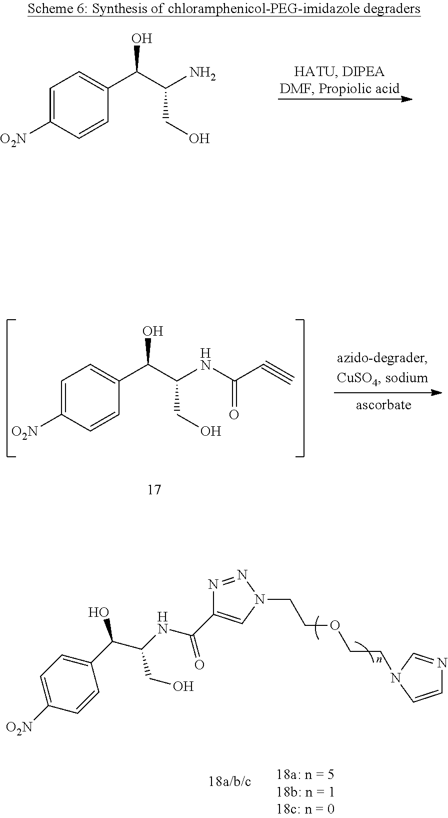

### Synthesis of chloramphenicol-6PEG-imidazole degrader (18a)

Propiolic acid (1.1 mmol) was dissolved in DMF (20 mL) under inert conditions and cooled to 0° C. HATU (2.0 mmol) and DIPEA (2.0 mmol) were added and the reaction mixture was stirred for 30 min at 0° C. (1R,2R)-(−)-2-Amino-1-(4-nitrophenyl)-1,3-propanediol (1.0 mmol) was added and the reaction mixture was stirred for 24 hours at room temperature. After filtration the crude mixture was directly used for the copper-click reaction. Azido-imidazole 3a (1.0 mmol), CuSO₄ (5 mol %), sodium ascorbate (0.2 mmol) were added, and the reaction mixture was stirred for another 24 hours at room temperature. After addition of toluene, the solvent was evaporated under reduced pressure. The crude product was purified via prep-HPLC. Yield: 10% (yellow oil).

¹H NMR (400 MHz, MeOD) δ 8.29 (s, 1H), 8.14 (d, J=8.8 Hz, 2H), 7.79 (s, 1H), 7.67 (d, J=8.7 Hz, 2H), 7.44 (s, 1H), 7.22 (s, 1H), 5.23 (d, J=2.6 Hz, 1H), 4.61-4.56 (m, 2H), 4.34 (s, 1H), 4.22 (t, J=4.9 Hz, 2H), 3.89-3.82 (m, 2H), 3.79-3.68 (m, 2H), 3.62-3.50 (m, 18H). MS: m/z for C₂₇H3₉N70₁₀: 621.3.

### Synthesis of chloramphenicol-2PEG-imidazole degrader (18b)

Propiolic acid (1.1 mmol) was dissolved in DMF (20 mL) under inert conditions and cooled to 0° C. HATU (2.0 mmol) and DIPEA (2.0 mmol) were added and the reaction mixture was stirred for 30 min at 0° C. (1R,2R)-(−)-2-Amino-1-(4-nitrophenyl)-1,3-propanediol (1.0 mmol) was added and the reaction mixture was stirred for 24 hours at room temperature. After filtration the crude mixture was directly used for the copper-click reaction. Azido-imidazole 3c (1.0 mmol), CuSO₄ (5 mol %), sodium ascorbate (0.2 mmol) were added, and the reaction mixture was stirred for another 24 hours at room temperature. After addition of toluene, the solvent was evaporated under reduced pressure. The crude product was purified via prep-HPLC. Yield: 10% (white solid).

¹H NMR (400 MHz, MeOD) δ 8.19 (s, 1H), 8.16 (d, J=8.8 Hz, 2H), 7.69 (d, J=8.6 Hz, 2H), 7.55 (s, 1H), 7.00 (s, 1H), 6.88 (s, 1H), 5.25 (d, J=2.8 Hz, 1H), 4.60 (dd, J=5.5, 4.5 Hz, 2H), 4.38 (ddd, J=7.0, 5.9, 2.9 Hz, 1H), 4.14 (dd, J=5.5, 4.4 Hz, 2H), 3.89 (dd, J=11.0, 7.0 Hz, 1H), 3.84 (t, J=5.1 Hz, 2H), 3.75 (dd, J=10.9, 5.9 Hz, 1H), 3.71 (t, J=4.9 Hz, 2H). MS: m/z for C₁₉H2₃N7Oe: 445.2.

### Synthesis of chloramphenicol-ethyl-imidazole degrader (18c)

Propiolic acid (1.1 mmol) was dissolved in DMF (20 mL) under inert conditions and cooled to 0° C. HATU (2.0 mmol) and DIPEA (2.0 mmol) were added and the reaction mixture was stirred for 30 min at 0° C. (1R,2R)-(−)-2-Amino-1-(4-nitrophenyl)-1,3-propanediol (1.0 mmol) was added and the reaction mixture was stirred for 24 hours at room temperature. After filtration the crude mixture was directly used for the copper-click reaction. Azido-imidazole 4 (1.0 mmol), CuSO₄ (5 mol %), sodium ascorbate (0.2 mmol) were added, and the reaction mixture was stirred for another 24 hours at room temperature. After addition of toluene, the solvent was evaporated under reduced pressure. The crude product was purified via prep-HPLC. Yield: 12% (white solid).

¹H NMR (400 MHz, MeOD) δ 8.14 (d, J=8.8 Hz, 2H), 8.03 (s, 1H), 7.64 (d, J=8.3 Hz, 2H), 7.43 (s, 1H), 6.97 (s, 1H), 6.93 (s, 1H), 5.21 (d, J=2.7 Hz, 1H), 4.82 (dd, J=6.8, 5.0 Hz, 2H), 4.58 (dd, J=6.7, 4.9 Hz, 2H), 4.31 (ddd, J=7.2, 5.9, 2.8 Hz, 1H), 3.84 (dd, J=10.9, 7.2 Hz, 1H), 3.69 (dd, J=10.9, 5.9 Hz, 1H). MS: m/z for C₁₇H1₉N7O₅: 401,1.

### Targetinq of G-Quadruplexes and Degradation of RNA

Two degraders were rationally designed to target the RNA G-quadruplex (rG4) by joining the known G4 binder pyridostatin with azido-imidazoles of different lengths (9A, 9B). The copper-induced azide-alkyne cycloaddition (CuAAC) used to join the two components tolerates a vast array of substrates and results in triazole, a bioisostere of an amide and a moiety well-tolerated in biological systems. We utilised the same strategy to synthesise CBX-PDS—a pyridostatin derivative that is known to selectively bind RNA, not DNA, G-quadruplexes. The two rG4 degraders and a binding control were tested in vitro and in cellular systems.

In vitro degradation of rG4 oligomers

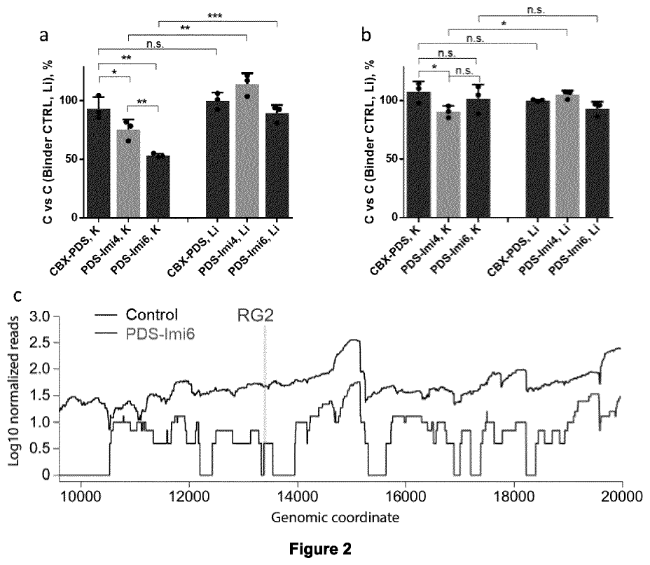

To show that our degraders could selectively degrade G4 structures, we examined their activity in presence of different oligomers and cations. We incubated our degraders with either an RNA oligomer corresponding to a rG4 structure in the 5′ UTR of NRAS mRNA or a mutated version thereof which was not capable of forming rG4. Additionally, we tested our molecules in presence of K+(a promoter of rG4 formation) and Li+(known to prevent rG4 formation). For the rG4 competent oligomer, degradation is observed only in the presence of K+, not Li+(FIG. 2a). This suggests that it is not sufficient for an oligomer to have a G-rich sequence to be targeted by these molecules; oligomers must form a G-quadruplex to be degraded. Strikingly, no degradation is observed with the perturbed rG4-incompetent NRAS sequence with either K+or Li+, which again shows that these degraders act specifically on rG4 structures (FIG. 2b). Moreover, no degradation was observed with CBX-PDS—a control molecule which contains the rG4 binder moiety PDS but not the degrader—which demonstrates that binding alone is not sufficient and a degrader moiety is necessary to achieve degradation (FIG. 2a, b). Interestingly, we observed that PDS-deg6 (9A), the degrader with a linker of 6 PEG subunits, was degrading RNA faster than PDS-deg4 (9B), with four PEG subunits. This might be a result of an extended reach of the longer linker. Altogether, these experiments demonstrate that our degraders specifically cleave rG4 species but not unfolded RNA regions.

In vitro degradation of SARS-CoV-2 genomic material

To provide evidence that rG4 degraders can degrade the genome of SARS-CoV-2 and gain insight into the mechanism of degradation, we have extracted viral RNA from VERO cells infected with SARS-CoV-2 and treated it with PDS-deg6 (9A), then analysed it via direct RNA sequencing. As SARS-CoV-2 genome has several putative rG4s sites (Zhao et al., 2021) and was shown to be tightly packed hence most of it in close proximity to an rG4 (Ziv et al., 2020), we hoped our degrader would induce wide-spread damage. Indeed, we observed substantial degradation across many regions of the genome, showing that our degrader is potent at damaging the genetic material of SARS-CoV-2 (FIG. 2c). This phenomenon suggests that our degraders should be capable of degrading and hence inactivating viral RNAs inside cells.

Anti-viral activity of rG4 degraders in vitro

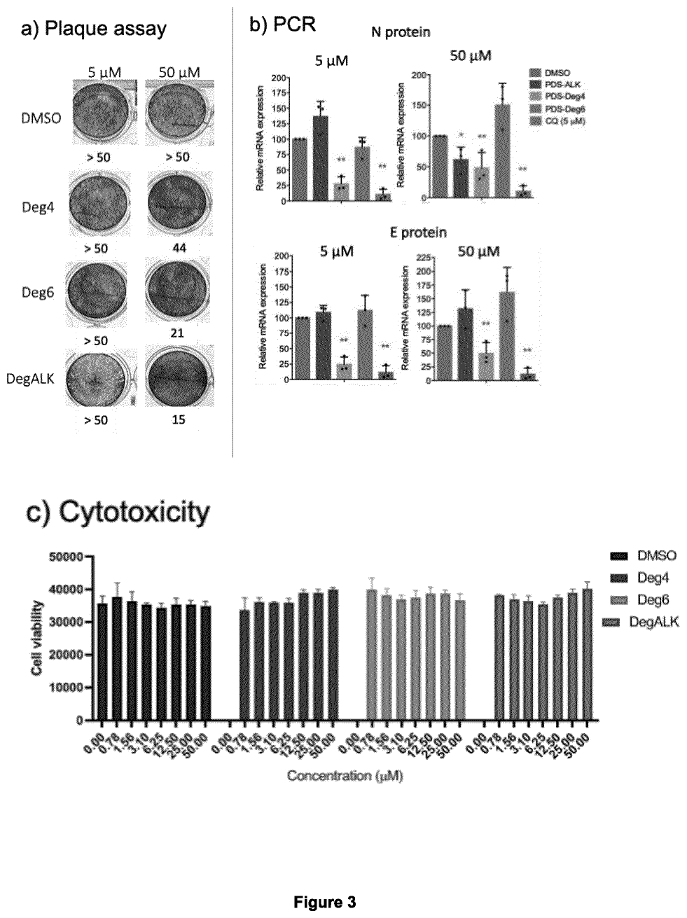

To test the antiviral the antiviral activity of the G4 degraders in vitro, 1 hour prior to infection, cells were incubated with the G4 degraders (PDS-deg6 (9A), PDS-deg4 (9B), and PDS-Alk (8): control molecule without the degrader) at 0.5 μM, 5 μM and 50 μM and chloroquine at 5 μM as a control (FIGS. 3a and 3b). Inhibition of viral growth was be measured by harvesting both the supernatant and cells at 24 hours after infection. Viral growth was assessed by measuring viral loads by plaque assay (in supernatant) and by PCR (in cells). The viability of cells after 24 hours of incubation with increasing concentrations of the G4-degrader (ranging from 0.05 μM to 50 μM) was assessed using a traditional cell viability kit (e.g. CellTiter Blue assay), according to the manufacturer's protocol.

We observed that G4 degraders successfully inhibit viral growth at 5 μM ad 50 μM (FIG. 3). PCR results showed that PDS-deg4 does not seem to inhibit viral replication, while PDS-deg6 (9A) inhibited 70% of viral replication at 5 μM in comparison to DMSO control (FIG. 3b). Importantly, none of the compounds showed cytotoxicity up to 50 μM (FIG. 3c).

Anti-viral activity of rG4 degraders in vivo

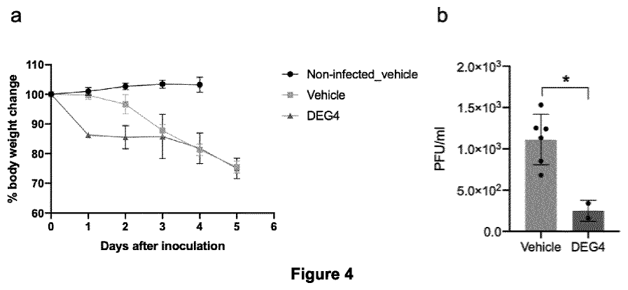

To assess the in vivo antiviral activity of G4 degraders, transgenic K18hACE2 mice (expressing hACE2 protein) were administered PDS-deg4 (9B) and PDS-deg6 (9A) at intranasally 25 mg/kg 40 minutes before infection, and again at 3 h and 18 h after infection (FIG. 4). Mice were infected with SARS-CoV-2 intranasally (with 2.5-5×10⁴ PFU/mouse in 50 μl of PBS on Day 0) and monitored on a daily basis for body weight, morbidity and mortality (found dead or euthanized in extremis) and clinical signs of infection. On day 5, all mice were sacrificed, and the left lung was collected for viral load quantification by plaque assay. Right lung, heart, liver, kidney and spleen were harvested for histopathological analysis.

Results showed that the administration of PDS-deg6 (9A) at 25 mg/kg was toxic to and these treated mice had to be sacrificed on Day 0. Organs were collected histopathological analysis. Mice administered with PDS-deg4 (9B) showed 10% loss of body weight in the first day after infection (FIG. 4a). However, body weight stabilized between Day 1 and Day 3, after which decreased again at the same rate as vehicle controls. Animals treated with PDS-deg4 (9B) showed a significant decrease in lung viral load (FIG. 4b).

This pilot showed that the administration of G4 degraders leads to decreased viral load in the lungs of SARS-CoV-2 infected k18hACE2 mice.

### Targeting of Pseudoknots and Degradation of RNA

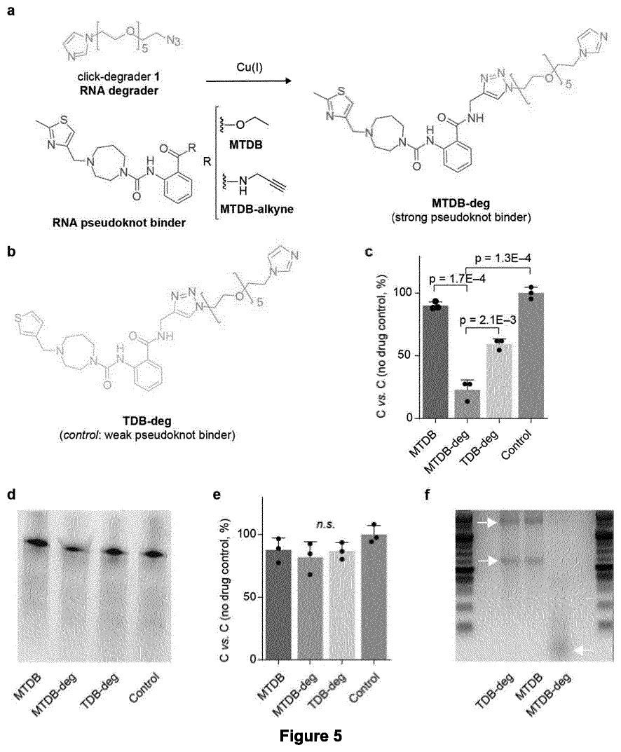

A non-covalent degrader molecule, MTDB-deg (16a), was rationally designed to target an RNA pseudoknot by joining the known pseudoknot binder MTDB with the azido-imidazole 3a (FIG. 5a). MTDB contains an ethyl ester moiety, which was exchanged for an amide to increase stability and for use as a handle for the attachment of the degrader. We used azido-imidazole 3a having a linker consisting of 6 PEG subunits, and which we previously found to be more effective than its shorter counterparts for RNA degradation of alkynyl-tagged RNAs (Mikutis et al., 2020). We chose CuAAC as the reaction to couple binder and degrader fragments because it is robust, easy to carry out, highly modular, and allows us to change the structures of the two fragments without altering the coupling step.

Selective degradation of the three-stemmed coronaviral pseudoknot

To validate our strategy, we tested our pseudoknot-degrader against an RNA 69-er with a sequence that corresponds to the coronaviral pseudoknot and thus is predicted to form it. We incubated the 69-er with MTDB-deg (16a) or one of the two control molecules—MTDB, the parent binder molecule that is not capable of degradation, or TDB-deg (16b), a degrader derived from 2-(4-(thiophen-3-ylmethyl)-[1,4]diazepane-1-carbonyl]-amino)-benzoic acid ethyl ester (TDB), which is closely related to MTDB but has a lower binding affinity towards the pseudoknot (FIG. 5b) (Park et al., 2011). After 3 h incubation with MTDB-deg (16a), the RNA pseudoknot had degraded significantly and just 23% remained intact relative to the non-treated sample, whereas inefficient degradation was seen with TDB-deg (16b) and no degradation was seen with MTDB (FIG. 5c,d). To illustrate that these molecules specifically bind and degrade the pseudoknot, we carried out the same experiment with an oligo similar to the pseudoknot but that harbours a heavily perturbed third stem that prevents it from properly forming the pseudoknot. As expected, none of the molecules affected the oligo stability (FIG. 5e). Thus, MTDB-deg (16a) was shown to bind and degrade the pseudoknot both efficiently and selectively.

To show that MTDB-deg (16a) is functional and can cut full-length coronaviral RNA, we incubated MTDB-deg (16a) and controls MTDB and TDB-deg (16b) with RNA extracted from SARS-CoV-2. Viral RNA was then analysed on agarose gels. We observed degradation only in the lane corresponding to MTDB-deg (16a) (FIG. 5f), further confirming that MTDB-deg (16a) does indeed degrade the native coronaviral pseudoknot, whereas the two control molecules do not, highlighting the specificity of the approach.

Specificity of coronaviral pseudoknot degradation by MTDB-deg

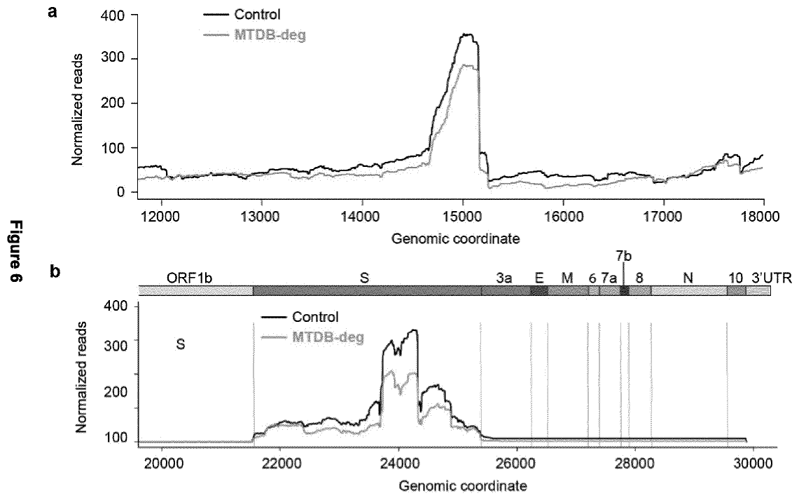

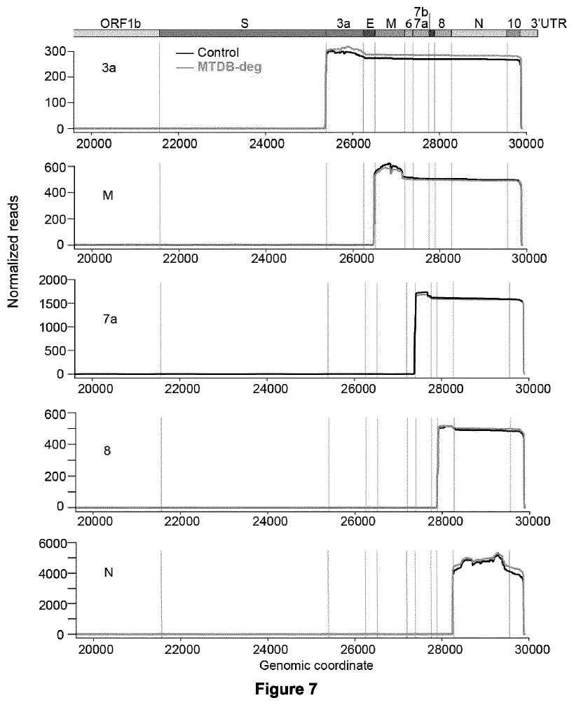

To further prove that MTDB-deg (16a) cuts the viral RNA, and to get a more precise picture of where the cut occurs, we analyzed the cut genomic RNA (gRNA) by direct RNA Nanopore sequencing. As expected, the region around the pseudoknot was affected the most (FIG. 6a). Interestingly, the pseudoknot flanks were more degraded than the pseudoknot itself. Indeed, a study on SARS-Cov-2 RNA interactome found that the region around the frameshifting element forms extensive short- and long-range interactions with the neighboring ORF1a and especially ORF1b; it is likely that the proximity of these elements to the pseudoknot enable MTDB-deg (16a) to efficiently cut them (FIG. 6a) (Ziv, et al., 2020). Interestingly, the only other structural element that was affected by the molecule was the S gene, which was shown to form long-range interactions with the ORF1b (Ziv, et al., 2020) and therefore is expected to be within reach of the degrader (FIG. 6b). Strikingly, none of the other sub-genomic regions were affected, which provides strong evidence for the specificity of MTDB-deg (16a) (FIG. 7). The above results provide strong proof of principle that MTDB-deg (16a) is a fully functional and selective degrader of the SARS-Cov-2 pseudoknot and its direct RNA-RNA interactome (Ziv, et al., 2020).

Efficiency and specificity of pseudoknot degradation in SARS-CoV-2 infected cells

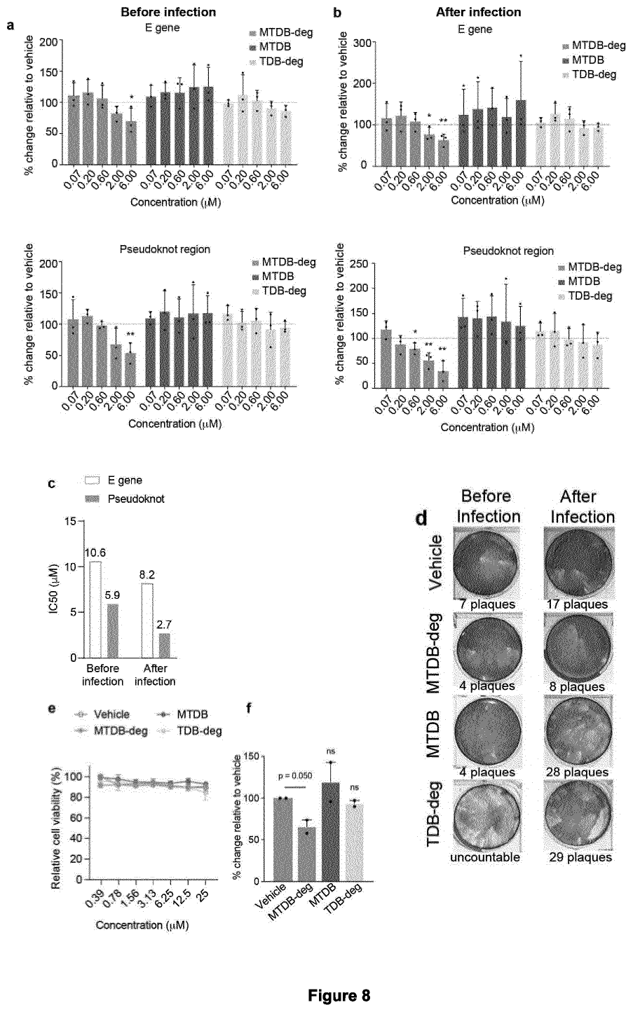

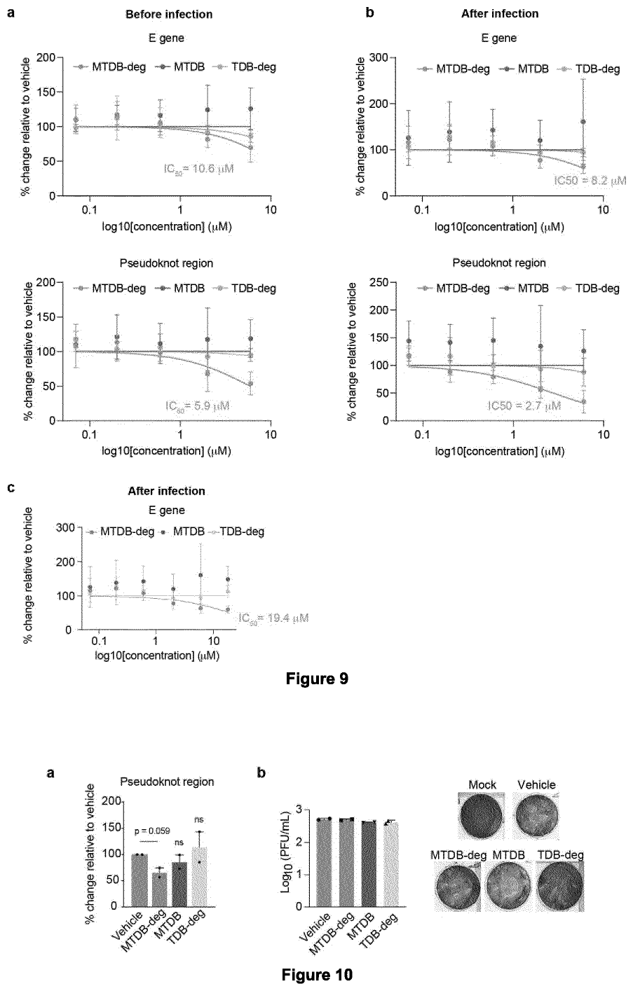

Having demonstrated the efficiency of MTDB-deg (16a) against the coronaviral pseudoknot in vitro, we investigated whether it could degrade the genome of SARS-CoV-2 in infected cells and thus prevent viral replication. We performed in vitro drug assays in which we measured SARS-CoV-2 replication on Vero CCL-81 cells. We observed that low-micromolar concentrations of MTDB-deg (16a) exhibited marked antiviral effects (FIG. 8a-c), with a significant reduction of coronaviral RNA in cells treated before (FIG. 8a; FIG. 9a) or after (FIG. 8b; FIG. 9b) infection. These results were supported by results from plaque assays (FIG. 8d). Importantly, control molecules MTDB and TDB-deg (16b) did not exhibit an antiviral effect, despite MTDB being known to disrupt frameshifting in SARS-CoV-2 (Kelly et al., 2020). Additionally, we found that none of the compounds were cytotoxic to host cells, which indicates that the observed effect on viral replication was a result of the specific antiviral activity of the compound (FIG. 8e). Curiously, the degrader was less active at concentrations higher than 6 μM (FIG. 9c), although no colloidal aggregation that could justify these readouts was seen in dynamic light scattering screens. Furthermore, the ability of the virus to recover from 24 h drug exposure was compromised in MTDB-deg (16a) treated samples, but not in samples treated with the control molecules MTDB and TDB-deg (16b) (FIG. 8f; FIG. 10a). Finally, no virucidal effect was observed when cell free virus was incubated with MTDB-deg (16a), MTDB or TDB-deg (16b), suggesting that the antiviral activity of MTDB-deg (16a) is mediated by direct inhibition of virus replication in host cells (FIG. 10b). Overall, the antiviral drug assays show that MTDB-degrader (16a) is an efficient antiviral agent against SARS-CoV-2 and is specific against coronaviral three-stemmed pseudoknots with irreversible impact.

Anti-viral activity of pseudoknot degraders in vivo

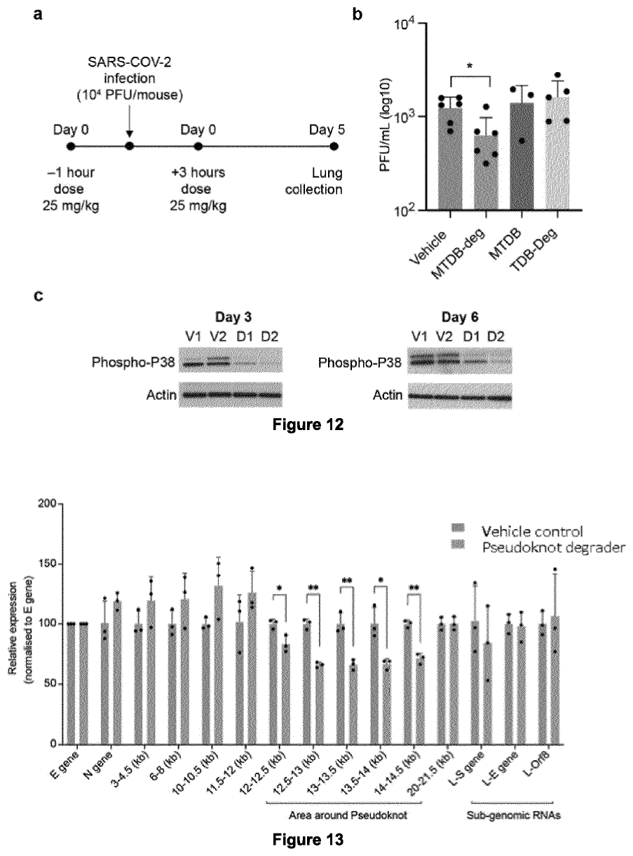

A SARS-CoV-2 mouse model of infection (transgenic K18-hACE2 mice) was used to determine the in vivo antiviral activity of the MTDB-deg 16a (FIG. 12a). Animals administered with MTDB-deg 16a (at 25 mg/kg) showed a significant reduction of lung viral load relative to the vehicle control group by plaque assay (FIG. 12b). Additionally, we investigated the in vivo antiviral potential of either MTDB-deg 16a or vehicle treatments using proteins extracted from lungs of K18-hACE2 transgenic mice on day 3 or day 6 post-infection. Reassuringly, we observed that at both timepoints of infection, the MTDB-deg 16a treated cohort showed a strong reduction in the phosphorylated levels of p38 (FIG. 12c), an important biomarker of SARS-CoV-2 infection and replication.

### Targeting of a Bacterial Ribosome

Three degraders were rationally designed to target the bacterial ribosome by joining the known ribosomal RNA binder chloramphenicol with azido-imidazoles of different lengths (18a, 18b, 18c). The chloramphenicol binder site was produced by peptide coupling of propargylic acid to (1R,2R)-(−)-2-Amino-1-(4-nitrophenyl)-1,3-propanediol using HATU and DIPEA, and followed by the coupling step using copper click chemistry. The copper-induced azide-alkyne cycloaddition (CuAAC) used to join the two components tolerates a vast array of substrates and results in triazole, a bioisostere of an amide and a moiety well-tolerated in biological systems. The three ribosomal degraders and a binding control were tested in vitro and in cellular systems.

In vitro degradation of E. coli ribozyme

The degradation activity was measured in an in vitro assay, targeting the E. coli ribosome. 200 μM ribozyme were incubated with degrader in different concentration, ranging from 15 mM to 0.47 mM, for 18 hours at 370C. Evaluation was made by agarose gel analysis.

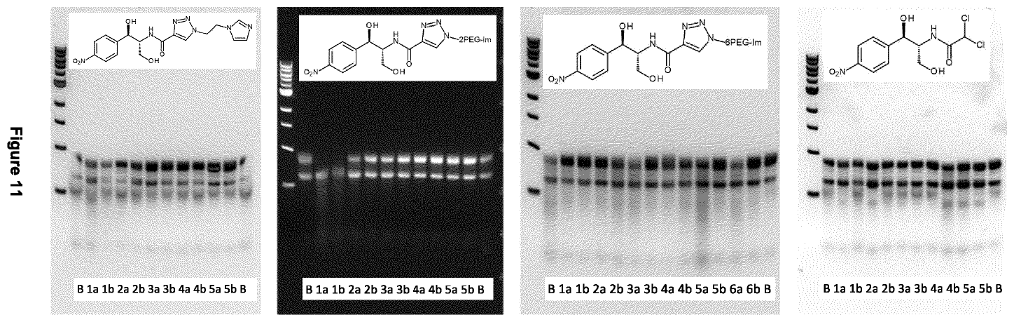

Best results were obtained using the PEG-2 linker (18b), where ribosome degradation could be observed at a concentration of 15 mM (FIG. 11).

### Additional Examples

### Materials and Methods

### Quantification of Viral Load by RT-qPCR

Cells from viral cultures were resuspended in RLT buffer and extracted using RNeasy Mini Kit. 500 ng of RNA was converted to cDNA using SuperScript™ VILO™ Master Mix. The levels of genomic and sub-genomic SARS-CoV-2 transcripts were analysed on a QuantStudio™ 5 real-time PCR machine (Applied Biosystems) using PowerUp™ SYBR™ Green Master Mix (Applied Biosciences) according to the manufacturer's instructions. All samples, including the template controls, were assayed in triplicates. The relative quantification of target gene expression was performed using the comparative cycle threshold (C_{T}) method. The primer sequences are listed in Table 1.

TABLE 1
Primer sequences used for RT-qPCR study on SARS-COV-2.

| Gene | Forward | Reverse |
|---|---|---|
| SARS-COV-2- | ACAGGTACGTTAATAGTTAATAGCGT | ATATTGCAGCAGTACGCACACA |
| E gene | | |
| SARS-COV-2- | GACCCCAAAATCAGCGAAAT | TCTGGTTACTGCCAGTTGAATCTG |
| N gene | | |
| SARS-COV-2- | GGAGGAGGTGTTGCAGGAGCCT | TGGGCCGACAACATGAAGACAGT |
| 3k-4.5k | | |
| SARS-COV-2- | AGTCAGAGGACGCGCAGGGAAT | TCTGTGTGGCCAACCTCTTCTGT |
| 6k-8k | | |
| SARS-COV-2- | ACCTCAGCTGTTTTGCAGAGTGGT | ACGTCATCAAGCCAAAGACCGT |
| 10k-10.5k | | |
| SARS-COV-2- | TGGGTGTTGGTGGCAAACCTTGT | ACTGGACACATTGAGCCCACAA |
| 11.5k-12k | | |
| SARS-COV-2- | TGCTTTCCATGCAGGGTGCTGT | AGCAACAGCCTGCTCATAAGCT |
| 12k-12.5k | | |
| SARS-COV-2- | TGCATCAGCATTGTGGGAAATCCA | ACCGGCAGCACAAGACATCTGT |
| 12.5k-13k | | |
| SARS-COV-2- | CCGCGAACCCATGCTTCAGTCA | CACGGTGTAAGACGGGCTGCAC |
| 13k-13.5k | | |
| SARS-COV-2- | ACACAATGGCAGACCTCGTCTATGC | AGCTTGGCGTACACGTTCACCT |
| 13.5k-14k | | |
| SARS-COV-2- | TGTGATGCCATGCGAAATGCTGGT | GGAACTCCACTACCTGGCGTGG |
| 14k-14.5k | | |
| SARS-COV-2- | AGCTCTTGGAGGTTCCGTGGCT | GGCTGTCCACCATGCGAAGTGT |
| 20k-21.5k | | |
| TRS-forward | CAAACCAACCAACTTTCGATCTCTTGTA | |
| SARS-COV-2- | | TTGTCAGGGTAATAAACACCACGT |
| S gene | | |
| SARS-COV-2- | | CGCCGTCAGGACAAGCAAAAGC |
| Orf8 | | |

Oligonucleotide preparation for binding studies

To prepare oligonucleotides for binding studies, oligonucleotides were dissolved at an appropriate buffer to a final concentration of 100 nM, then incubated at 95° C. with shaking for 5 min. Samples were allowed to cool at room temperature for at least 1 h before further dilutions/treatments.

Fluorescence quenching assays

Cy5-tagged oligonucleotide corresponding to G4 structure found on 5′UTR of NRAS mRNA (final concentration 50 nM) were dissolved in 20 mM HEPES pH 7.4 buffer supplemented with KCl (100 mM) and MgCl₂ (10 mM) and plated on a 96-well plate. Oligonucleotides were treated with various concentrations of PDS family ligands as indicated, ranging from 5 nM to 10 μM, or a water vehicle control, followed by 30 minute incubation at 4° C. Fluorescence corresponding to Cy5 fluorophore was then measured for each oligonucleotide-small molecule/vehicle control combination using a plate reader (BMG CLARIOstar) at 25.0° C. For all the molecules, 12 serial dilutions with a dilution ratio 1:1 were used, with the highest concentration tested being 10 μM. Fluorescence was normalised to a vehicle control and binding curves for each molecule were obtained via a sigmoidal fit by setting the Hill Slope coefficient to 1.

Micro-scale thermophoresis (MST) assays

FAM-tagged oligonucleotide (final concentration 50 nM) was dissolved in 20 MM HEPES pH 7.4 buffer supplemented with KCl (100 mM) and EDTA (10 mM). Oligonucleotide solutions were then treated with various concentrations of small molecules and analysed via MST according to the manufacturer's instructions (NanoTemper Monolith NT.115). For these MST measurements the following programme was used: 5 seconds laser off, 30 seconds laser on, 5 second laser off; 20% LED (blue) power, 30% MST power; measurements were carried out at 25.0° C. For MTDB-Deg, TDB-Deg and Click-degrader 1, 12 serial dilutions with a dilution ratio 5:3 were used, with the highest concentration tested being 8 mM. For MTDB, 12 serial dilutions with a dilution ratio 3:1 were used, with the highest concentration tested being 250 μM. Binding curves for each molecule were obtained via a sigmoidal fit by setting the Hill Slope coefficient to 1.

TABLE 2
Sequences of oligonucleotides used for
binding assays.

| RNA species | Sequence |
|---|---|
| G4 on NRAS | 5′-Cy5-UGUGGGAGGGGGGGGUCUGGGUGC-3′ |
| 5′UTR | |
| Pseudoknot | 5′-FAM-UUUGCGGUGUAAGUGCAGCCCGUCUUA |
| | CACCGUGCGGCACAGGCACUAGUACUGAUGUCGU |
| | AUACAGGGCUUU-3′ |
| Perturbed | 5′-FAM-UUUGCGGUGUAAGUGCAGCCCGUCUUA |
| | CACCGUGAAUAUAUGGCACUAGUACUGAUGUCGU |
| | AUACAGGGCUUU-3′ |

Pseudoknot degradation by MTDB-deg

This example investigates the interaction between MTDB-deg and a pseudoknot in an infection model of SARS-CoV-2. VERO cells infected with SARS-CoV-2 were treated with either with MTDB-deg or a vehicle control, followed by RNA extraction and qPCR analysis on 14 loci of the SARS-CoV-2 genome so to get a broad genomic coverage and reveal which segments are affected the most.

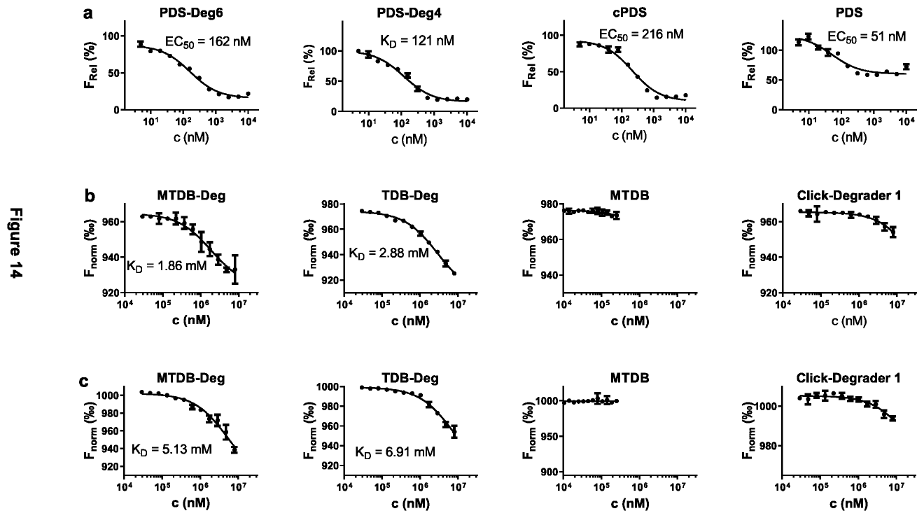

The results are shown in FIG. 13. These are in broad agreement with in vitro experiments, demonstrating that MTDB-deg affects the flanks of the pseudoknot area but not any other region of the full length genomic or sub-genomic SARS-CoV-2 RNA. This demonstrates that MTDB-deg is a functional and selective degrader of the SARS-CoV-2 pseudoknot and its direct RNA-RNA interactome.

Binding affinity determination

Having prepared two series of compounds, the effect of functionalisation with degraders on binding affinity and selectivity was tested next. PDS is a known fluorescence-quencher, thus a fluorescence quenching assay was used to evaluate compounds derived from this scaffold (Di Antonio et al., 2012).

PDS and its derivatives were incubated with a Cy5-functionalised oligomer corresponding to a G-quadruplex in NRAS 5′UTR, a well-established G4 model. As fluorescence quenching is proximity-induced, the signal corresponding to Cy5 is quenched upon PDS binding. Based on this assay, we found that the parent compound PDS binds with EC₅₀ of 51 nM (FIG. 14a), with the PDS derivatives having a slightly lower affinity compared to the parent compound. This is because likely to be because non-derivatised PDS has an additional amine group on the pyridine core which has been replaced with a triazole in the derived compounds; this positively charged amine group might be forming additional interactions with the negatively charged phosphate backbone.

MTDB and derived ligands do not quench fluorescence, thus microscale thermophoresis (MST) was utilised to evaluate the binding affinities for this family of RNA binders. In an MST workflow, a FAM-functionalised oligomer corresponding to the coronaviral pseudoknot is incubated with one of the binders, with the fluorescence of the bound oligomer exhibiting different temperature-dependence compared to the non-bound oligomer.

To investigate the binding selectivity of MTDB, assays on an oligonucleotide corresponding to a perturbed pseudoknot were carried out in addition to the pseudoknot. The perturbed pseudoknot has one of its stems changed into a random sequence of the same length and thus is no longer able to form the full pseudoknot structure.

MTDB was found to be not soluble enough for K_{D} determination using MST although the trend of the curve indicated noticeable binding at concentrations 187.5 μM and above (FIG. 14b). No appreciable binding was observed for the mutated (perturbed) pseudoknot (FIG. 14c). The other two molecules that were tested, MTDB-deg and TDB-deg, were found to be more soluble in aqueous media and thus concentrations ranging from 29 μM to 8 mM were tested.

Using the MST approach, it was found that MTDB-deg and TDB-deg have K_{D} values of 1.86 and 2.88 mM, respectively (FIG. 14b). When carried out on the perturbed pseudoknot, the K_{D} values fell to 5.13 mM and 6.91 mM, respectively (FIG. 14c). This indicates that the binder-degrader molecules are selective yet weak binders. Similarly to MTDB, the shape of the MTDB-deg binding curve indicates noticeable binding with concentrations above 134 μM, indicating that functionalization with the degrader did not compromise the binding affinity of this molecule.

The degrader-linker component 3a (Click-Degrader 1) was also tested for bind to the pseudoknot oligonucleotides. With the concentrations tested, the binding was too weak to obtain a K_{D} value, suggesting that this molecule is a much poorer binder than molecules derived from MTDB or TDB (FIG. 14b,c).

### REFERENCES

A number of publications are cited above in order to more fully describe and disclose the invention and the state of the art to which the invention pertains. Full citations for these references are provided below. The entirety of each of these references is incorporated herein.
Bobbin, et al., Annual Review of Pharmacology and Toxicology, 2016, Vol. 56, pp. 103-122.
Cox, et al., Science, 2017, Vol. 358, pp. 1019-1027.
Di Antonio et al., Angew. Chem. int. Edit., 2012, Vol 51, pp. 11073-11078
Gasiunas, et al., Proc. Natl. Acad. Sci. U.S.A., 2012, Vol. 109, E2579-E2586.
Jinek, et al., Science, 2012, Vol. 337, pp. 816-821.
Kelly et al., J. Biol. Chem., 2020, Vol. 295, pp. 10741-10748.
Li. Bioinformatics, 2018, Vol. 34, pp. 3094-3100.
Mikutis et al., ACS Cent. Sci., 2020, Vol. 6, pp. 2196-2208.
Park et al., J. Am. Chem. Soc., 2011, Vol. 133, pp. 10094-10100.
Santos et al., Pharmaceuticals, 2021, Vol. 14, No. 769
Tzelepis, et al., Cell Reports, 2016, Vol. 17, pp. 1193-1205.
Zamore, et al., Cell, 2000, Vol. 101, pp. 25-33.
Zhao et al., Angew. Chem. int. Ed., 2021, 60 (1), 432-438.
Ziv, et al., “Mol. Cell., 2020, Vol. 80, pp. 1067-1077.

1. A method for cleaving a target nucleic acid molecule, the method comprising:
contacting the target nucleic acid molecule with a bifunctional molecule of formula (I) or a salt or solvate thereof:
C-L-B (I)
where —C is a cleavage group that is imidazole, optionally substituted with 1 to 3 C₁.e alkyl groups, -L- is a linker and —B is a non-covalent binding group, such that the bifunctional molecule non-covalently binds to the target nucleic acid molecule, and;
allowing the bifunctional molecule to cleave the target nucleic acid molecule bound thereto.

2. The method of claim 1, wherein the non-covalent binding group is not a polynucleotide group.

3. The method of claim 1 or 2, wherein the non-covalent binding group has molecular weight of 1,000 kDa or less.

4. The method of any preceding claim, wherein the non-covalent binding group binds to a secondary or tertiary structure within the target nucleic acid.

5. The method of claim 4, wherein the non-covalent binding group binds to a quadruplex or a pseudoknot.

6. The method of claim 5, wherein the bifunctional molecule binds to the quadruplex or pseudoknot with a dissociation constant (k_{D}) of 10,000 nM or less.

7. The method of claim 5 or 6, wherein the bifunctional molecule binds to the quadruplex or pseudoknot with a selectivity of 5:1.

8. The method of any preceding claim, wherein the non-covalent binding group is selected from formulae (B-I) to (B-III): 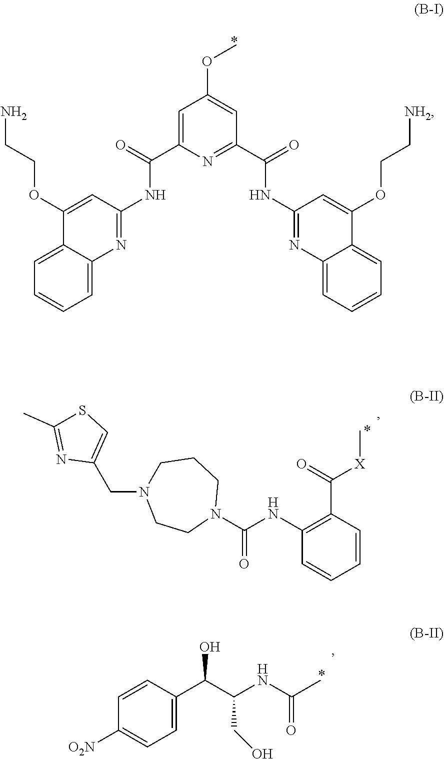
where:
X is O or NH; and
* is the attachment point with the linker.

9. The method of any preceding claim, wherein the cleavage group is selected form the groups represented by formula (C-I) to (C-III): 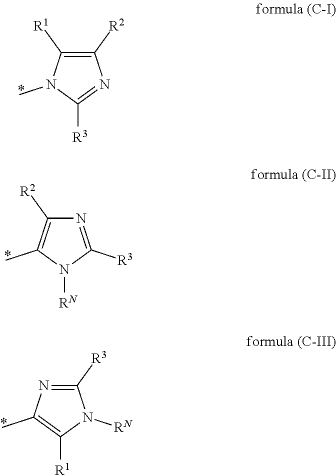
where:
R¹, R² and R³ each independently represent a hydrogen atom or a C₁0.6 alkyl group;
R^{N} represents a hydrogen atom or a C₁0.6 alkyl group; and
* represents the attachment position with the remainder of the molecule (typically the linker unit L).

10. The method of claim 9, wherein the cleavage group is a group represented by formula (C-I).

11. The method of claim 10, wherein the cleavage group is unsubstituted imidazole.

12. The method of any preceding claim, wherein the linker comprises a polyalkylene glycol group.

13. The method of any preceding claim, wherein the target nucleic acid molecule is an RNA molecule.

14. The method of any preceding claim, wherein the target nucleic acid molecule is contacted with the bifunctional molecule within a cell.

15. A method for identifying a secondary or tertiary structure within a target nucleic acid molecule, the method comprising:
providing first and second populations of nucleic acid molecules, each population comprising the target nucleic acid molecule;
introducing into the first population of nucleic acid molecules a bifunctional molecule of formula (I) or a salt or solvate thereof:
C-L-B (I)
where —C is a cleavage group that is imidazole, optionally substituted with 1 to 3 C₁₋₆ alkyl groups, -L- is a linker and —B is a non-covalent binding group, such that the bifunctional molecule non-covalently binds to the target nucleic acid molecule;
allowing the bifunctional molecule to cleave the target nucleic acid molecule present in the first population; and
identifying nucleic acid molecules which are present in a reduced amount in the first population relative to the second population.

16. The method of claim 15, wherein the non-covalent binding group is not a polynucleotide group.

17. The method of claim 15 or 16, wherein the non-covalent binding group has molecular weight of 1,000 kDa or less.

18. The method of any of claims 15 to 17, wherein the non-covalent binding group binds to a secondary or tertiary structure within the target nucleic acid.

19. The method of claim 18 wherein the non-covalent binding group binds to a quadruplex or pseudoknot.

20. The method of claim 19, wherein the bifunctional molecule binds to the quadruplex or pseudoknot with a dissociation constant (k_{D}) of 10,000 nM or less.

21. The method of claim 19 or 20, wherein the bifunctional molecule binds to the quadruplex or pseudoknot with a selectivity of 5:1.

22. The method of any of claims 15 to 21, wherein the non-covalent binding group is selected from formulae (B-I) to (B-III): 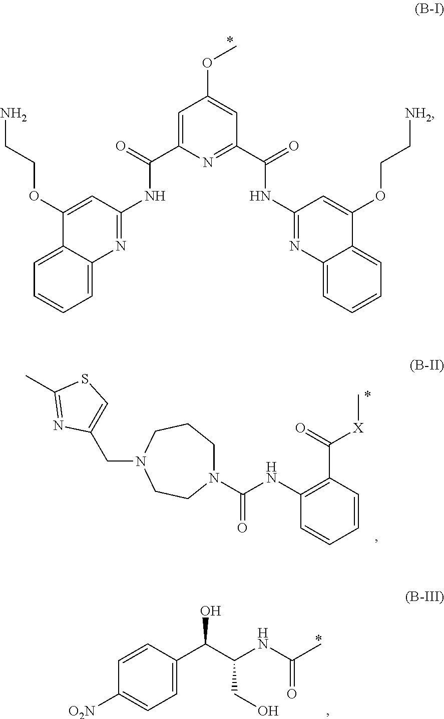
where:
X is O or NH; and
* is the attachment point with the linker.

23. The method of any of claims 15 to 22, wherein the cleavage group is selected form the groups represented by formula (C-I) to (C-III): 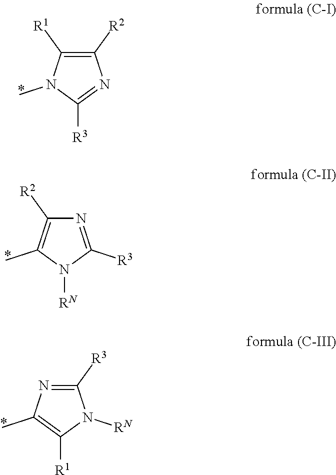
where:
R¹, R² and R³ each independently represent a hydrogen atom or a C₁0.6 alkyl group;
R^{N} represents a hydrogen atom or a C₁0.6 alkyl group; and
* represents the attachment position with the remainder of the molecule (typically the linker unit L).

24. The method of claim 23, wherein the cleavage group is a group represented by formula (C-I).

25. The method of claim 24, wherein the cleavage group is unsubstituted imidazole.

26. The method of any of claims 15 to 25, wherein the linker comprises a polyalkylene glycol group.

27. The method of any of claims 15 to 26, wherein the target nucleic acid molecule is an RNA molecule.

28. The method of any of claims 15 to 27, wherein the target nucleic acid molecule is contacted with the bifunctional molecule within a cell.

29. A bifunctional molecule of formula (I), or a pharmaceutically acceptable salt or solvate thereof, for use in a method of treatment:
C-L-B (I)
where —C is a cleavage group that is imidazole, optionally substituted with 1 to 3 C₁₋₆ alkyl groups, -L- is a linker and —B is a non-covalent binding group that binds to a target nucleic acid molecule.

30. The bifunctional molecule for use of claim 29, wherein the non-covalent binding group is not a polynucleotide group.

31. The bifunctional molecule for use of claim 29 or 30, wherein the non-covalent binding group has molecular weight of 1,000 kDa or less.

32. The bifunctional molecule for use of any of claims 29 to 31, wherein the non-covalent binding group binds to a secondary or tertiary structure within a target nucleic acid.

33. The bifunctional molecule for use of any of claims 29 to 32 wherein the non-covalent binding group binds to a quadruplex or pseudoknot.

34. The bifunctional molecule for use of claim 33, wherein the bifunctional molecule binds to the quadruplex or pseudoknot with a dissociation constant (k_{D}) of 10,000 nM or less.

35. The bifunctional molecule for use of claim 33 or 34, wherein the bifunctional molecule binds to the quadruplex or pseudoknot with a selectivity of 5:1.

36. The bifunctional molecule for use of any of claims 29 to 35, wherein the non-covalent binding group is selected from formulae (B-I) to (B-III): 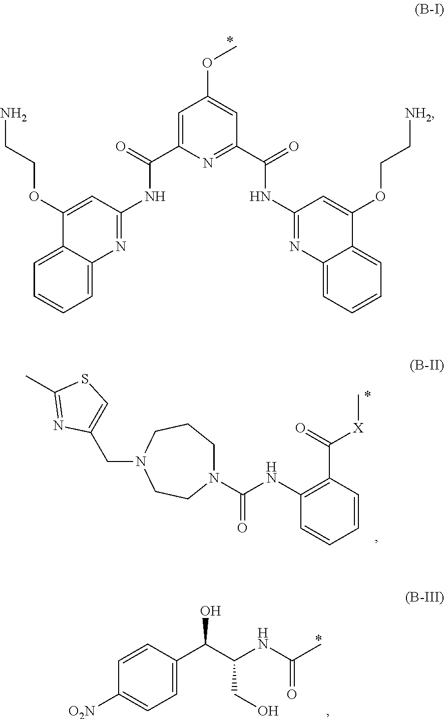
where:
X is O or NH; and
* is the attachment point with the linker.

37. The bifunctional molecule for use of any of claims 29 to 36, wherein the cleavage group is selected form the groups represented by formula (C-I) to (C-III): 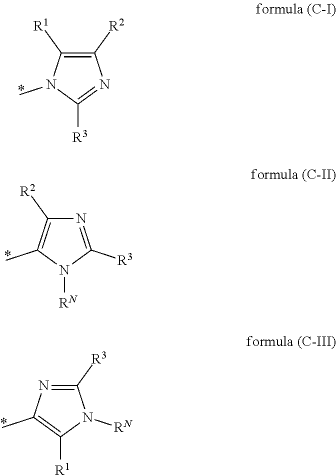
where:
R¹, R² and R³ each independently represent a hydrogen atom or a C₁0.6 alkyl group;
R^{N} represents a hydrogen atom or a C₁0.6 alkyl group; and
* represents the attachment position with the remainder of the molecule (typically the linker unit L).

38. The bifunctional molecule for use of claim 37, wherein the cleavage group is a group represented by formula (C-I).

39. The bifunctional molecule for use of claim 38, wherein the cleavage group is unsubstituted imidazole.

40. The bifunctional molecule for use of any of claims 29 to 39, wherein the linker comprises a polyalkylene glycol group.

41. The bifunctional molecule for use of any of claims 29 to 40, wherein the target nucleic acid molecule is an RNA molecule.

42. The bifunctional molecule for use of any of claims 29 to 41, wherein the treatment is treatment of a bacterial or viral infection.

43. The bifunctional molecule for use of claim 42, wherein the treatment is treatment of an infection with an RNA virus.

44. The bifunctional molecule for use of claim 43, wherein the virus is a coronavirus.

45. The bifunctional molecule for use of any of claims 29 to 41, wherein the treatment is treatment of a respiratory tract infection, a urinary tract infection, or gastroenteritis.

46. A bifunctional molecule of formula (I), or a salt or solvate thereof:
C-L-B (I)
where —C is a cleavage group that is imidazole, optionally substituted with 1 to 3 C₁₋₆alkyl groups, -L- is a linker and —B is a non-covalent binding group that binds to a nucleic acid molecule.

47. The bifunctional molecule of claim 46, wherein the non-covalent binding group is not a polynucleotide group.

48. The bifunctional molecule of claim 46 or 47, wherein the non-covalent binding group has molecular weight of 1,000 kDa or less.

49. The bifunctional molecule of any of claims 46 to 48, wherein the non-covalent binding group binds to a quadruplex or pseudoknot.

50. The bifunctional molecule of any of claims 46 to 49, wherein the non-covalent binding group is selected from formulae (B-I) to (B-III): 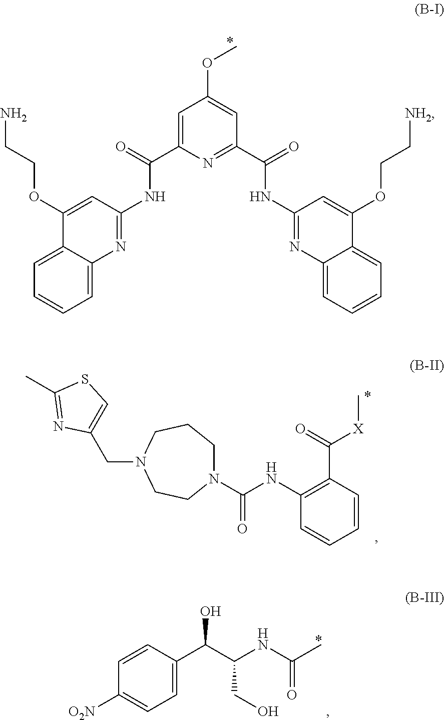
where:
X is O or NH; and
* is the attachment point with the linker.

51. The bifunctional molecule of any of claims 46 to 50, wherein the cleavage group is selected form the groups represented by formula (C-I) to (C-III): 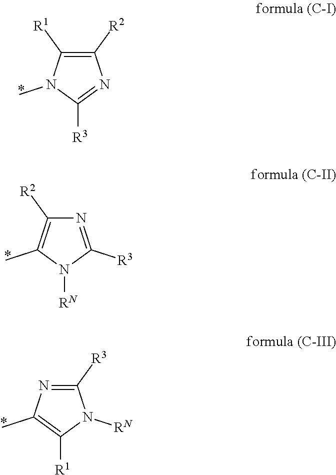
where:
R¹, R² and R³ each independently represent a hydrogen atom or a C₁₋₆ alkyl group;
R^{N} represents a hydrogen atom or a C₁₋₆ alkyl group; and
* represents the attachment position with the remainder of the molecule (typically the linker unit L).

52. The bifunctional molecule of claim 51, wherein the cleavage group is a group represented by formula (C-I).

53. The bifunctional molecule of claim 52, wherein the cleavage group is unsubstituted imidazole.

54. The bifunctional molecule of any of claims 46 to 53, wherein the linker comprises a polyalkylene glycol group.

55. The bifunctional molecule of any of claims 46 to 54, wherein the linker comprises a group represented by formula (L-1): 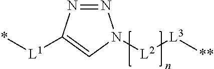
where:
L¹ is a covalent bond or a C₁₋₂ alkylene group
L² is a C₁₋₆ alkylene group or a C₁₋₆ heteroalkene group
L³ is a C₁₋₆ alkylene group
n is 1 to 8;
* is the attachment point with the non-covalent binding group (—B); and
** is the attachment point with the cleavage group (—C).

56. The bifunctional molecule of claim 55, wherein:
L¹ is methylene;
L² is ethylene oxide;
L³ is ethylene; and
n is 2 to 5.

57. The bifunctional molecule of claim 46 selected from compounds of formula Deg-I to Deg-V:

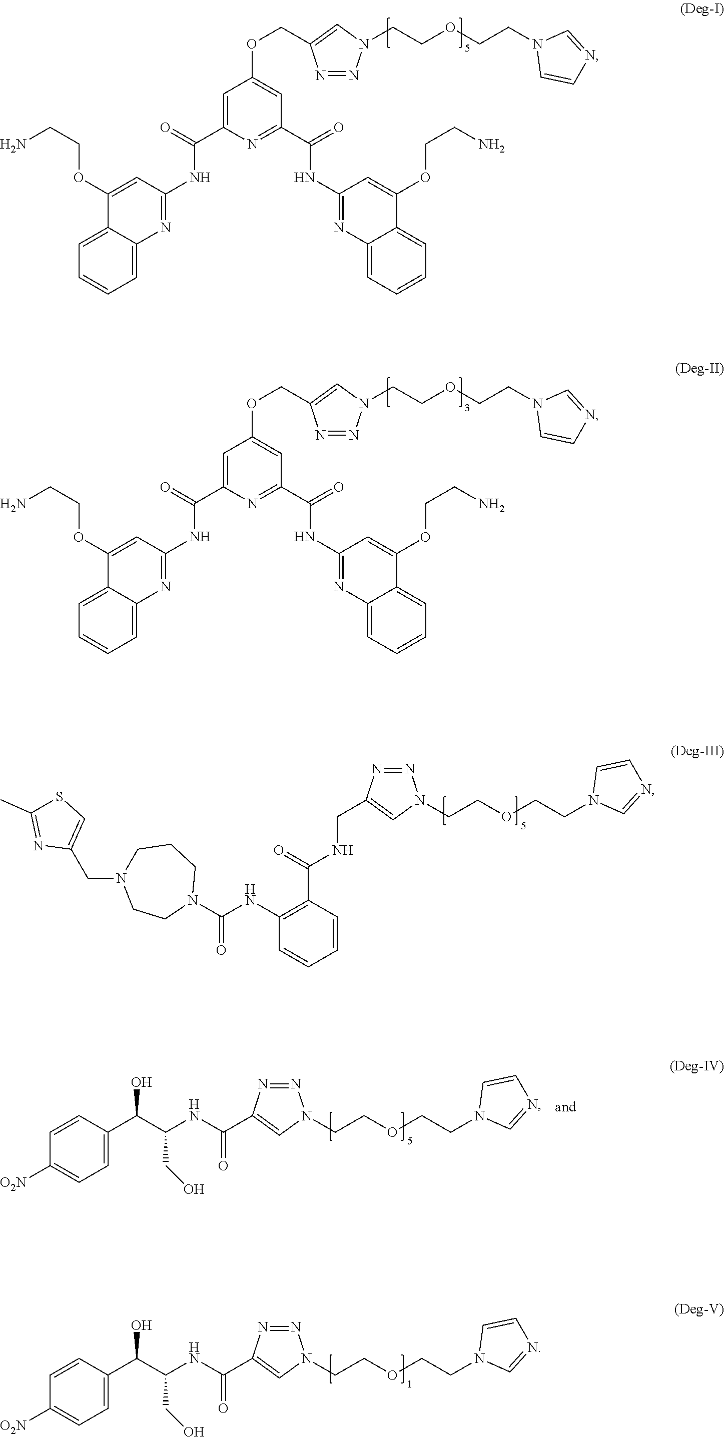

58. The method of claim 5 or claim 19, wherein the bifunctional molecule binds to the quadruplex or pseudoknot with a dissociation constant (k_{D}) of 10 mM or less.

59. The method of claim 5 or claim 19, wherein the bifunctional molecule binds to the pseudoknot with a dissociation constant (k_{D}) of 10 mM or less.

60. The bifunctional molecule for use of claim 33, wherein the bifunctional molecule binds to the quadruplex or pseudoknot with a dissociation constant (k_{D}) of 10 mM or less.

61. The bifunctional molecule for use of claim 33, wherein the bifunctional molecule binds to the pseudoknot with a dissociation constant (k_{D}) of 10 mM or less.

62. The bifunctional molecule for use of any of claims 29 to 41, 60 and 61, wherein the treatment is treatment of cancer.

63. The bifunctional molecule of claim 49, wherein the bifunctional molecule binds to the quadruplex or pseudoknot with a dissociation constant (k_{D}) of 10 mM or less, such as 10,000 nM or less.

64. The bifunctional molecule of claim 63, wherein the bifunctional molecule binds to the quadruplex with a dissociation constant (k_{D}) of 10,000 nM or less.

65. The bifunctional molecule of claim 63, wherein the bifunctional molecule binds to the pseudoknot with a dissociation constant (k_{D}) of 10 mM or less.


SEQUENCE LISTING (32 sequences)

SEQ ID NO: 1 - DNA, 26 bp, synthetic construct
  source 1..26: mol_type = other DNA
acaggtacgttaatagttaatagcgt

SEQ ID NO: 2 - DNA, 22 bp, synthetic construct
  source 1..22: mol_type = other DNA
atattgcagcagtacgcacaca

SEQ ID NO: 3 - DNA, 20 bp, synthetic construct
  source 1..20: mol_type = other DNA
gaccccaaaatcagcgaaat

SEQ ID NO: 4 - DNA, 24 bp, synthetic construct
  source 1..24: mol_type = other DNA
tctggttactgccagttgaatctg

SEQ ID NO: 5 - DNA, 22 bp, synthetic construct
  source 1..22: mol_type = other DNA
ccgcgaacccatgcttcagtca

SEQ ID NO: 6 - DNA, 22 bp, synthetic construct
  source 1..22: mol_type = other DNA
cacggtgtaagacgggctgcac

SEQ ID NO: 7 - DNA, 20 bp, synthetic construct
  source 1..20: mol_type = other DNA
gtaacccgttgaaccccatt

SEQ ID NO: 8 - DNA, 20 bp, synthetic construct
  source 1..20: mol_type = other DNA
ccatccaatcggtagtagcg

SEQ ID NO: 9 - DNA, 22 bp, synthetic construct
  source 1..22: mol_type = other DNA
ggaggaggtgttgcaggagcct

SEQ ID NO: 10 - DNA, 23 bp, synthetic construct
  source 1..23: mol_type = other DNA
tgggccgacaacatgaagacagt

SEQ ID NO: 11 - DNA, 22 bp, synthetic construct
  source 1..22: mol_type = other DNA
agtcagaggacgcgcagggaat

SEQ ID NO: 12 - DNA, 23 bp, synthetic construct
  source 1..23: mol_type = other DNA
tctgtgtggccaacctcttctgt

SEQ ID NO: 13 - DNA, 24 bp, synthetic construct
  source 1..24: mol_type = other DNA
acctcagctgttttgcagagtggt

SEQ ID NO: 14 - DNA, 22 bp, synthetic construct
  source 1..22: mol_type = other DNA
acgtcatcaagccaaagaccgt

SEQ ID NO: 15 - DNA, 23 bp, synthetic construct
  source 1..23: mol_type = other DNA
tgggtgttggtggcaaaccttgt

SEQ ID NO: 16 - DNA, 22 bp, synthetic construct
  source 1..22: mol_type = other DNA
actggacacattgagcccacaa

SEQ ID NO: 17 - DNA, 22 bp, synthetic construct
  source 1..22: mol_type = other DNA
tgctttccatgcagggtgctgt

SEQ ID NO: 18 - DNA, 22 bp, synthetic construct
  source 1..22: mol_type = other DNA
agcaacagcctgctcataagct

SEQ ID NO: 19 - DNA, 24 bp, synthetic construct
  source 1..24: mol_type = other DNA
tgcatcagcattgtgggaaatcca

SEQ ID NO: 20 - DNA, 22 bp, synthetic construct
  source 1..22: mol_type = other DNA
accggcagcacaagacatctgt

SEQ ID NO: 21 - DNA, 25 bp, synthetic construct
  source 1..25: mol_type = other DNA
acacaatggcagacctcgtctatgc

SEQ ID NO: 22 - DNA, 22 bp, synthetic construct
  source 1..22: mol_type = other DNA
agcttggcgtacacgttcacct

SEQ ID NO: 23 - DNA, 24 bp, synthetic construct
  source 1..24: mol_type = other DNA
tgtgatgccatgcgaaatgctggt

SEQ ID NO: 24 - DNA, 22 bp, synthetic construct
  source 1..22: mol_type = other DNA
ggaactccactacctggcgtgg

SEQ ID NO: 25 - DNA, 22 bp, synthetic construct
  source 1..22: mol_type = other DNA
agctcttggaggttccgtggct

SEQ ID NO: 26 - DNA, 22 bp, synthetic construct
  source 1..22: mol_type = other DNA
ggctgtccaccatgcgaagtgt

SEQ ID NO: 27 - DNA, 28 bp, synthetic construct
  source 1..28: mol_type = other DNA
caaaccaaccaactttcgatctcttgta

SEQ ID NO: 28 - DNA, 24 bp, synthetic construct
  source 1..24: mol_type = other DNA
ttgtcagggtaataaacaccacgt

SEQ ID NO: 29 - DNA, 22 bp, synthetic construct
  source 1..22: mol_type = other DNA
cgccgtcaggacaagcaaaagc

SEQ ID NO: 30 - RNA, 24 nt, synthetic construct
  source 1..24: mol_type = other RNA
tgtgggaggggcgggtctgggtgc

SEQ ID NO: 31 - RNA, 73 nt, synthetic construct
  source 1..73: mol_type = other RNA
tttgcggtgtaagtgcagcccgtcttacaccgtgcggcacaggcactagtactgatgtcgtatacagggcttt

SEQ ID NO: 32 - RNA, 73 nt, synthetic construct
  source 1..73: mol_type = other RNA
tttgcggtgtaagtgcagcccgtcttacaccgtgaatatatggcactagtactgatgtcgtatacagggcttt